(19) United States
(12) Patent Application Publication
(10) Pub. No.: US 2016/0325113 A1
(43) Pub. Date: Nov. 10, 2016

(54) Phototherapy Devices and Methods Comprising Optionally Substituted Quinquiesphenyl Compounds

(71) Applicant: NITTO DENKO CORPORATION, Osaka (JP)

(72) Inventors: Sazzadur Rahman Khan, San Diego, CA (US); Shijun Zheng, San Diego, CA (US); Sheng Li, Vista, CA (US); Amane Mochizuki, Carlsbad, CA (US); Keisaku Okada, San Diego, CA (US)

(21) Appl. No.: 15/213,253

(22) Filed: Jul. 18, 2016

Related U.S. Application Data
(63) Continuation-in-part of application No. 14/394,064, filed on Oct. 10, 2014.
(60) Provisional application No. 61/624,119, filed on Apr. 13, 2012.

(51) Int. Cl. A61N 5/06; C09K 11/06; C09K 11/02; A61K 41/00; H01L 51/00

(52) U.S. Cl. CPC A61N 5/062; A61K 41/0061; A61K 41/0071; H01L 51/0061; H01L 51/0072; H01L 51/0052; C09K 11/025; C09K 11/06; H01L 51/0085; H01L 51/0037; C09K 2211/185; A61N 2005/0653; A61N 2005/0663; H01L 51/5231

(57) ABSTRACT

Methods and devices related to the treatment of diseases using phototherapy are described. Some embodiments provide an organic light-emitting diode device, such as a light-emitting device for phototherapy, comprising Ring System 1, Ring System 2, Ring System 3, Ring System 4 or Ring System 5. Methods of treating disease with phototherapy are also described.

### CROSS REFERENCE TO RELATED APPLICATIONS

This application is a continuation-in-part of U.S. application Ser. No. 14/394,064 filed on Oct. 10, 2014, which is the U.S. national phase of International Patent Application No. PCT/US2013/36482 filed on Apr. 12, 2013, which claims priority to U.S. Provisional Patent Application No. 61/624,119 filed on Apr. 13, 2012, the entire disclosures of which are incorporated by reference.

### BACKGROUND

Phototherapy may be useful in treating a number of medical conditions. However, light sources such as lasers, which may be used for phototherapy, may be expensive, difficult to transport, and not suitable for home or outpatient treatment. Therefore, there may be a need for alternative sources of light for phototherapy which may be less expensive and more portable.

### SUMMARY

Some embodiments relate to organic light-emitting devices which may be used for phototherapy. These devices typically comprise an organic light-emitting diode, such as an organic light-emitting diode comprising an anode, a cathode, and an organic light-emitting layer disposed between the anode and the cathode. In some embodiments, the organic light-emitting layer may include an optionally substituted quinquiesphenyl compound, such as a compound described herein. These optionally substituted quinquiesphenyl compounds may be used as emissive compounds, or may act as host compounds in conjunction with other emissive materials.

In phototherapy, light from a light-emitting device may be used to provide a therapeutic effect by contact between the emitted light and tissue of a mammal or a person. The light may directly provide a therapeutic effect or may activate a photosensitive compound on or in the tissue. An activated photosensitive compound may directly provide a therapeutic effect, or may indirectly provide a therapeutic effect by reaction to another chemical species which has a therapeutic effect, or by some other mechanism.

Some light-emitting devices that may used in phototherapy comprise a light-emitting layer comprising a compound comprising optionally substituted Ring System 1, optionally substituted Ring System 2, optionally substituted Ring System 3, optionally substituted Ring System 4, or optionally substituted Ring System 5.

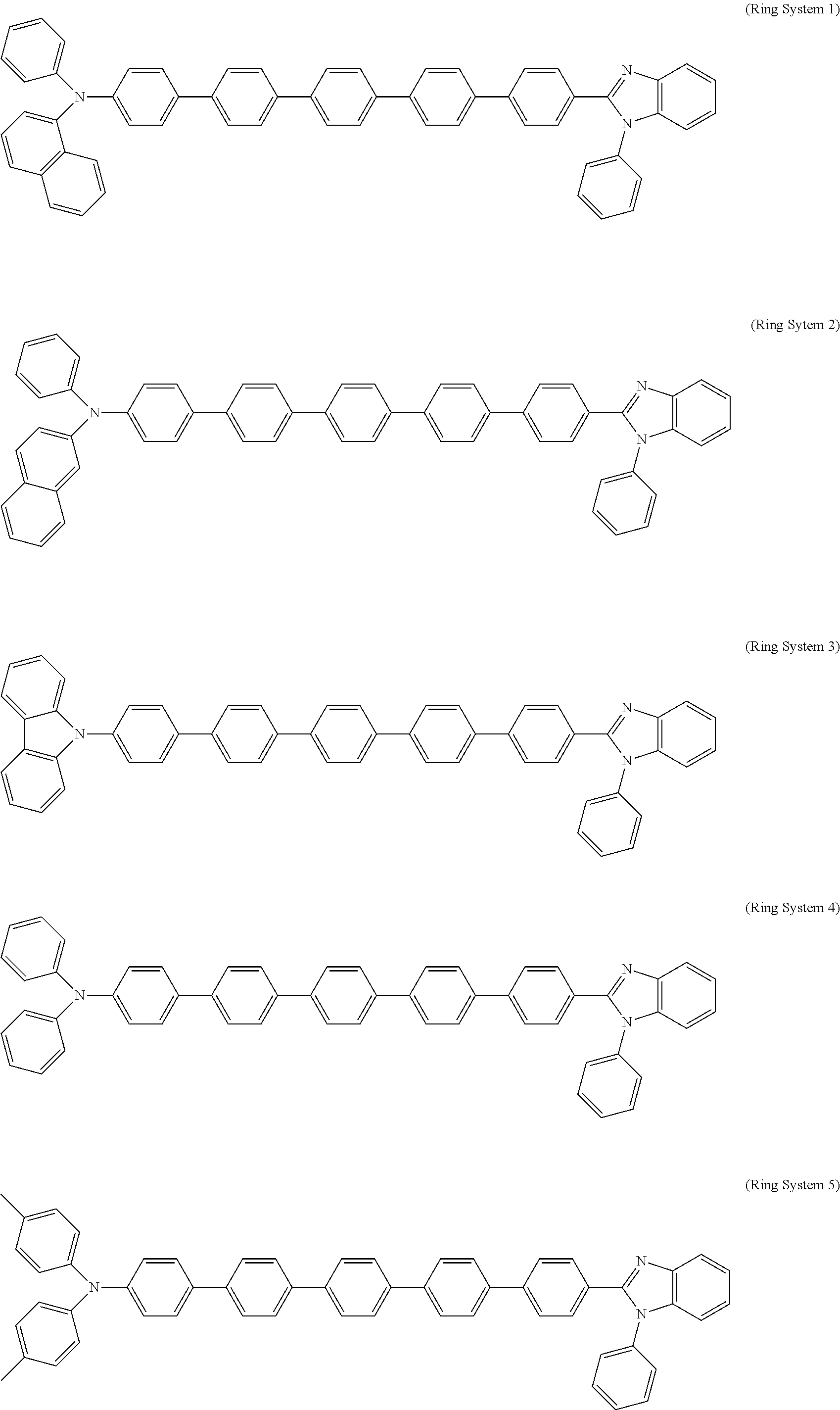

wherein the device is configured to emit an effective amount of light to provide a therapeutic effect to a mammal, wherein the therapeutic effect is provided by contact between at least a portion of the emitted light and the mammal.

Some embodiments include a phototherapy system comprising: a light-emitting device described herein and a wound dressing.

Some embodiments include a method of treating a disease, comprising: administering a photosensitive compound to a tissue of a mammal in need thereof and exposing at least a portion of the tissue to light from a device described herein; wherein at least a portion of the photosensitive compound is activated by at least a portion of the light from the device to which the tissue is exposed, to thereby treat the disease.

Some embodiments include a phototherapy system comprising: a device described herein and a photosensitive compound, wherein the photosensitive compound is suitable for administration to a tissue of a mammal in need of phototherapy, and wherein the device is configured to emit light of a wavelength which can activate at least a portion of the photosensitive compound when the photosensitive compound is in the tissue.

These and other embodiments are described in greater detail below.

### BRIEF DESCRIPTION OF THE DRAWINGS

FIG. 1 is a schematic diagram of an embodiment of a bottom emissive device.

FIG. 2 is a schematic diagram of an embodiment of a top emissive device.

FIG. 3 is a schematic of some embodiments which further include a controller, a processor, and an optional detector.

FIG. 4 is the electroluminescence spectrum of device-A.

FIG. 5 is a plot of current density/brightness vs. voltage curve of device-A

FIG. 6 is a plot of current efficiency/power efficiency vs. luminance of device-A.

FIG. 7 the electroluminescence spectrum of the embodiment of device-B.

FIG. 8 is a plot of current density/brightness vs. voltage curve of device-B.

FIG. 9 is a plot of current efficiency/power efficiency vs. luminance of device-B.

FIG. 10 is a plot of light output and surface temperature as a function of input current of device B.

FIG. 11 is a schematic representation of ex-vivo efficacy study of an embodiment of a light-emitting device.

FIGS. 12A and 12B show the image of Chinese Hamster Ovarian Cancer cells before and after the light irradiation from an embodiment of a light-emitting device.

FIG. 13 shows cell viability (%) data with respect to the varying dose of irradiation in a 1 mM ALA solution.

FIG. 14 is a plot of efficiency and lifetime of the devices of example 9.

### DETAILED DESCRIPTION

Unless otherwise indicated, when a chemical structural feature such as aryl is referred to as being “optionally substituted,” it includes a feature that may have no substituents (i.e. be unsubstituted) or may have one or more substituents. A feature that is “substituted” has one or more substituents. The term “substituent” has the ordinary meaning known to one of ordinary skill in the art. A substituent generally includes at least 1, or 1-5, 1-10, 1-20, or 1-30, atoms independently selected from: C, N, O, S, P, Si, F, Cl, Br, I, or a combination thereof, and may include hydrogen atoms. In some embodiments a substituent may comprise at least one of: C, N, O, S, P, Si, F, Cl, Br, and I, and/or may have a molecular weight of: at least about 15; and/or less than about 500, about 300, about 200, about 150, about 100, about 75, or about 50. In some embodiments, the substituent has at least 1 carbon atom or at least 1 heteroatom, and has about 0-10 carbon atoms and about 0-5 heteroatoms independently selected from: N, O, S, F, Cl, Br, I, and combinations thereof. In some embodiments, each substituent consists of: about 0-20 carbon atoms; about 0-47 hydrogen atoms; 0, 1, 2, 3, 4, or 5 oxygen atoms; 0, 1, or 2 sulfur atoms; 0, 1, 2, or 3 nitrogen atoms; 0 or 1 silicon atoms; 0, 1, 2, 3, 4, 5, 6, or 7 fluorine atoms; 0, 1, 2, or 3 chlorine atoms; 0, 1, 2, or 3 bromine atoms; and 0, 1, 2, or 3 iodine atoms. Examples include, but are not limited to, alkyl, alkenyl, alkynyl, carbazolyl, cycloalkyl, cycloalkenyl, cycloalkynyl, aryl, diarylamino, heteroaryl, heteroalicyclyl, aralkyl, heteroaralkyl, (heteroalicyclyl)alkyl, hydroxy, protected hydroxy, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, and amino, including mono- and di-substituted amino groups, and the protected derivatives thereof.

In some embodiments, substituents may include, but are not limited to, C₁₋₁₀ alkyl such as methyl, ethyl, propyl isomers (e.g. n-propyl and isopropyl), cyclopropyl, butyl isomers, cyclobutyl isomers (e.g. cyclobutyl, methylcyclopropyl, etc.), pentyl isomers, cyclopentyl isomers, hexyl isomers, cyclohexyl isomers, heptyl isomer, cycloheptyl isomers, etc; alkoxy such as —OCH₃, —OC₂H₅, —OC₃H₇, —OC₄H₉, —OC₅H₁₁, —OC₆H₁₃, —OC₇H₁₅, etc.; halo, such as F, Cl, Br, I, etc.; haloalkyl, including perfluoroalkyl such as —CF₃, —C₂F₅, —C₃F₇, —C₄F₉, etc.; C₁₋₁₀ acyl such as formyl, acetyl, benzoyl, etc.; C₁₋₁₀ amides attaching at the carbonyl or nitrogen atom such as —NCOCH₃, —CONHCH₂, etc.; C₁₋₁₀ esters attaching at the carbonyl or oxygen atom such as —OCOCH₃, —OC₂CH₂, etc.; carbamates attaching at the nitrogen atom or oxygen atom; cyano; cyanate; isocyanate; nitro; etc.

In some embodiments, the substituents may be selected from: F, Cl, C₁₋₆ alkyl, —O—C₁₋₆ alkyl, CN, NO₂, and CF₃.

In some embodiments, the compounds may consist essentially of: Ring System 1, Ring System 2, Ring System 3, Ring System 4, or Ring System 5, each without substituents, or Ring System 1, Ring System 2, Ring System 3, Ring System 4, or Ring System 5, each with one or more substituents on the Ring System. In some embodiments, Ring System 1, Ring System 2, Ring System 3, Ring System 4, or Ring System 5 may each have 1, 2, 3, 4, 5, 6, 7, 8, 9, or 10 substituents.

A quinquiesphenyl ring structure is shown below.

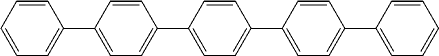 [SMILES: c1ccc(-c2ccc(-c3ccc(-c4ccc(-c5ccccc5)cc4)cc3)cc2)cc1]

The term “work function” has the ordinary meaning known to one of ordinary skill in the art. In some embodiments, the “work function” of a metal refers to a measure of the minimum energy required to extract an electron from the surface of the metal.

The term “high work function metal” has the ordinary meaning known to one of ordinary skill in the art. In some embodiments, a “high work function metal” includes a metal or alloy that easily injects holes and typically has a work function greater than or equal to 4.5.

The term “low work function metal” has the ordinary meaning known to one of ordinary skill in the art. In some embodiments, a “low work function metal” includes a metal or alloy that easily loses electrons and typically has a work function less than 4.3.

The compounds and compositions described herein can be incorporated into light-emitting devices in various ways. Some light-emitting devices may comprise an organic component disposed between an anode and a cathode. An organic component may comprise the compounds and/or compositions described herein. For example, a compound described herein may be a host in an emissive layer, a host in a layer that is not an emissive layer, or may be a light-emitting component in an emissive layer.

An organic component may comprise one or more layers comprising organic materials such as an emissive layer, a hole-transport layer, an electron-transport layer, a hole-injection layer, an electron injection layer, etc. In some embodiments, the compounds described may be used as an emissive compound, as an ambipolar host in an organic light-emitting diode emissive layer, or both. Some compounds disclosed herein may provide well balanced hole-transport and electron-transport mobility in a device, which may lead to a simpler device structure with high quantum efficiency and low turn-on voltage. For example, in some organic light-emitting diodes or devices comprising the compounds described herein may not have a hole-transporting layer or an electron-transporting layer. In some embodiments, these compounds may have high electrochemical stability, high thermal stability, a high glass transition temperature (Tg), and high photostability. Thus, these compounds may provide an OLED device with a longer lifetime than existing OLED devices.

Some devices comprising a compound described herein may be schematically represented in FIG. 1. Such a device comprises the following layers in the order given: an anode 5, a hole-injection layer 10, a hole-transport layer 15, a light-emitting layer 20, an electron-transport layer 30, and a cathode 35.

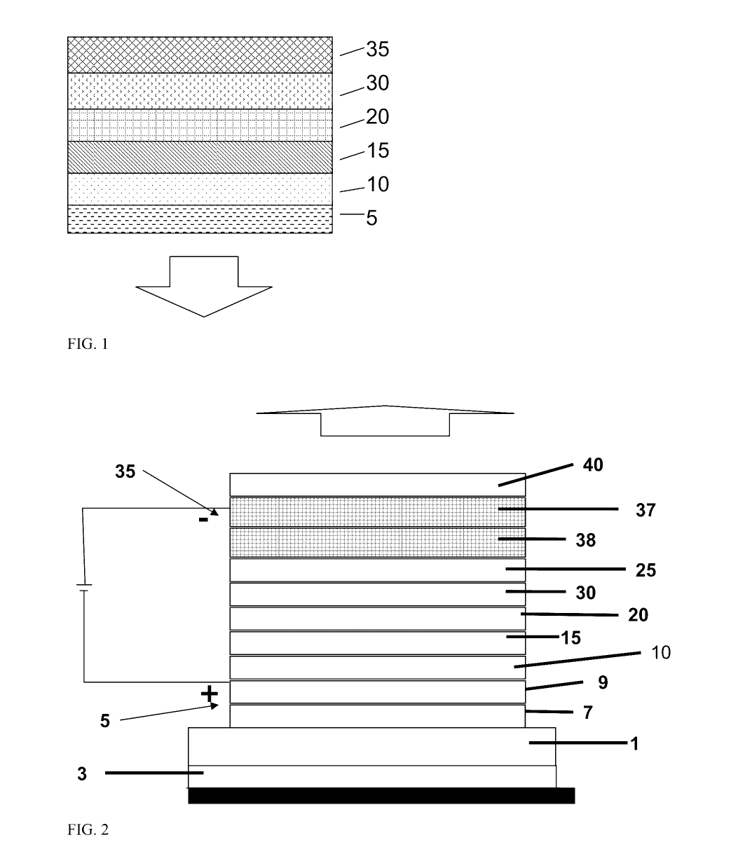

Some embodiments may have a structure represented schematically by FIG. 2. A light-emitting layer 20 is disposed between an anode 5 and cathode 35. The cathode 35 may comprise two cathode sublayers: a first cathode sublayer 37, and a second cathode sublayer 38 disposed between the first cathode sublayer 37 and the light-emitting layer 20. The anode 5 may comprise two anode sublayers: a first anode sublayer 7, and a second anode sublayer 9 disposed between the first anode sublayer 7 and the light-emitting layer 20. An optional electron-injecting layer 25 may be disposed between the cathode 35 or the second cathode sublayer 38 and the light-emitting layer 20. An optional electron-transport layer 30 may be disposed between the light-emitting layer 20 and the cathode 35, the second cathode sublayer 38, or the electron-injecting layer 25. An optional hole-injecting layer 10 may be disposed between the light-emitting layer 20 and the anode 5 or the second anode sublayer 9. An optional hole-transport layer 15 may be disposed between the hole-injecting layer 10 and the light-emitting layer 20. The anode 5 may optionally be disposed on a substrate 1, and the substrate 1 may optionally be disposed on a heat dissipation layer 3. A capping layer 40 may optionally be disposed on the cathode 35.

An anode layer, e.g. anode 5, may comprise a conventional material such as a metal, mixed metal, alloy, metal oxide or mixed-metal oxide, or a conductive polymer. Examples of suitable metals include Group 10, Group 11, and Group 12 transition metals. If the anode layer is to be light-transmitting, mixed-metal oxides of Groups 12, Group 13, and Group 14 metals or alloys thereof, such as zinc oxide, tin oxide, indium zinc oxide (IZO) or indium-tin-oxide (ITO) may be used. The anode layer may include an organic material such as polyaniline, e.g., as described in “Flexible light-emitting diodes made from soluble conducting polymer,” Nature, vol. 357, pp. 477-479 (11 Jun. 1992). Examples of suitable high work function metals include but are not limited to Au, Pt, indium-tin-oxide (ITO), or alloys thereof. In some embodiments, the anode layer can have a thickness in the range of about 1 nm to about 1000 nm.

A first anode sublayer, e.g. first anode sublayer 7 may comprise Al, Ag, Ni, or a combination thereof. The thickness of a first anode sublayer may vary. For example, a first anode sublayer may have thickness of: about 10 nm to about 100 nm, about 10 nm to about 70 nm, about 40 nm to about 60 nm, about 10 nm, about 50 nm, about 70 nm, about 100 nm, or any thickness in a range defined by, or between, any of these values.

A second anode sublayer, e.g. second anode sublayer 9 may comprise Al, Ag, Au, or a combination thereof. The thickness of a second anode sublayer may also vary. For example, a second anode sublayer may have a thickness of: about 5 nm to about 200 nm, about 10 nm to about 100 nm, about 30 nm to about 70 nm, about 25 nm, about 40 nm, about 50 nm, to about 200 nm, or any thickness in a range defined by, or between, any of these values.

In some embodiments, a first anode sublayer may comprise Ag and/or a second anode sublayer may comprise Al.

A cathode layer, e.g. cathode 35, may include a material having a lower work function than the anode layer. Examples of suitable materials for the cathode layer include those selected from alkali metals of Group 1, Group 2 metals, Group 11, Group 12, and Group 13 metals including rare earth elements, lanthanides and actinides, materials such as aluminum, indium, calcium, barium, samarium and magnesium, and combinations thereof. Li-containing organometallic compounds, LiF, and Li₂O may also be deposited between the organic layer and the cathode layer to lower the operating voltage. Suitable low work function metals include but are not limited to Al, Ag, Mg, Ca, Cu, Mg/Ag, LiF/Al, CsF, CsF/Al or alloys thereof. In some embodiments, the cathode layer can have a thickness in the range of about 1 nm to about 1000 nm.

A first cathode sublayer, e.g. first cathode sublayer 37 may comprise alkali metals of Group 1, Group 2 metals, Group 12 metals including rare earth elements, lanthanides and actinides, materials such as aluminum, indium, calcium, barium, samarium and magnesium, and combinations thereof. In some embodiments, a first cathode sublayer comprises Al, Ag, Au, Cu, Mg/Ag, or alloys thereof.

The thickness of a first cathode sublayer may vary. For example, a second cathode sublayer may have a thickness of about 0.1 nm, about 1 nm, about 2 nm, about 4 nm, about 5 nm, about 6 nm, about 10 nm, about 12 nm, about 16, about 20 nm, about 50 nm, or any thickness in a range defined by, or between, any of these values. In some embodiments, a second cathode sublayer may have a thickness in a range of about 0.1 nm to about 50 nm, about 1 nm to about 20 nm, about 5 nm to about 20 nm, or about 16 nm.

A second cathode sublayer, e.g. second cathode sublayer 38 may comprise alkali metals of Group 1, Group 2 metals, Group 12 metals including rare earth elements, lanthanides and actinides, materials such as aluminum, indium, calcium, barium, samarium and magnesium, and combinations thereof. In some embodiments, a second cathode sublayer comprises Mg, Ca, Mg/Ag, LiF/AI, CsF, CsF/AI or alloys thereof.

The thickness of a second cathode sublayer may vary. For example, a second cathode sublayer may have thickness of about 0.1 nm to about 50 nm, about 0.1 nm to about 10 nm, about 0.5 nm to about 2 nm, about 0.1 nm, about 1 nm, about 2 nm, about 4 nm, about 5 nm, about 6 nm, about 10 nm, about 12 nm, about 20 nm, about 50 nm, or any thickness in a range defined by, or between, any of these values.

In some embodiments, a first cathode sublayer comprises Mg/Ag and/or a second cathode sublayer comprises Mg. In some embodiments, a first cathode sublayer is about 16 nm thick and/or a second cathode sublayer is about 1 nm thick.

A light-emitting layer, e.g. light-emitting layer 20, may comprise a light-emitting component, and optionally, a host. For example, a light-emitting layer may consist essentially of a light-emitting component. Alternatively, a light-emitting layer may comprise a light-emitting component and other components such as a host. A host may comprise a compound described herein, a hole-transport material, an electron-transport material, and/or an ambipolar material. In some devices, a light emitting component may be about 0.1% to about 10%, about 1% to about 5%, or about 3% of the mass of the light-emitting layer.

If present, the amount of a host in a light-emitting layer may vary. In one embodiment, the amount of a host in a light-emitting layer may be in the range of from about 1% to nearly 100% by weight of the light-emitting layer. In another embodiment, the amount of a host in a light-emitting layer may be about 90% to about 99% by weight, or about 97% by weight, of the light-emitting layer.

A light-emitting component may be a fluorescent and/or a phosphorescent compound, including but not limited to a compound disclosed herein. In some embodiments, the light-emitting component comprises a phosphorescent material.

Some non-limiting examples of compounds which may form part or all of a light-emitting component include iridium coordination compounds such as: Bis[2-(2′-benzothienyl)-pyridinato-N,C3′] iridium (III)(acetylacetonate), Bis[(2-phenylquinolyl)-N,C2′]iridium (III) (acetylacetonate); Bis[(1-phenylisoquinolinato-N,C2′)]iridium (III) (acetylacetonate); Bis[(dibenzo[f, h]quinoxalino-N,C2′)iridium (III)(acetylacetonate), Tris(2,5-bis-2′-(9′,9′-dihexylfluorene)pyridine)iridium (III), Tris[1-phenylisoquinolinato-N,C2′]iridium (III), Tris-[2-(2′-benzothienyl)-pyridinato-N,C3′]iridium (III), Tris[1-thiophen-2-ylisoquinolinato-N,C3′]iridium (III), and Tris[1-(9,9-dimethyl-9H-fluoren-2-yl)isoquinolinato-(N,C3′)iridium (III)), etc.

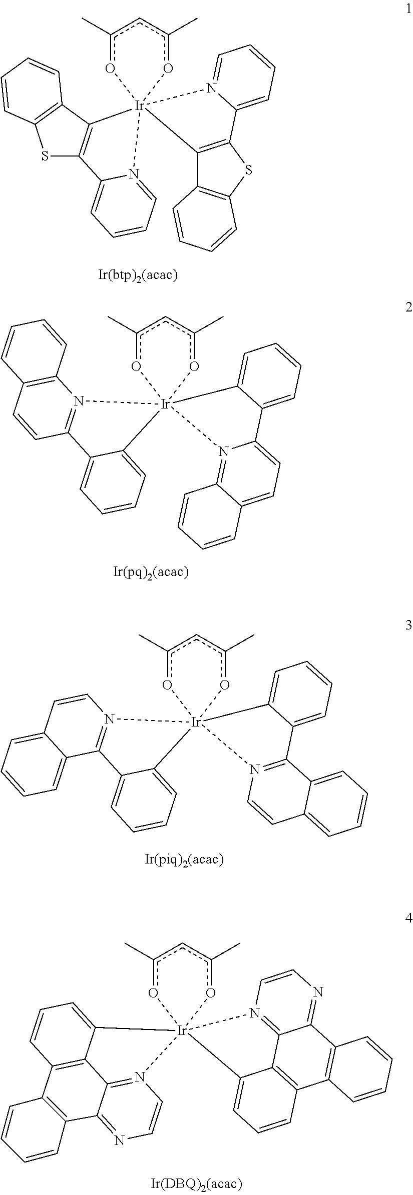

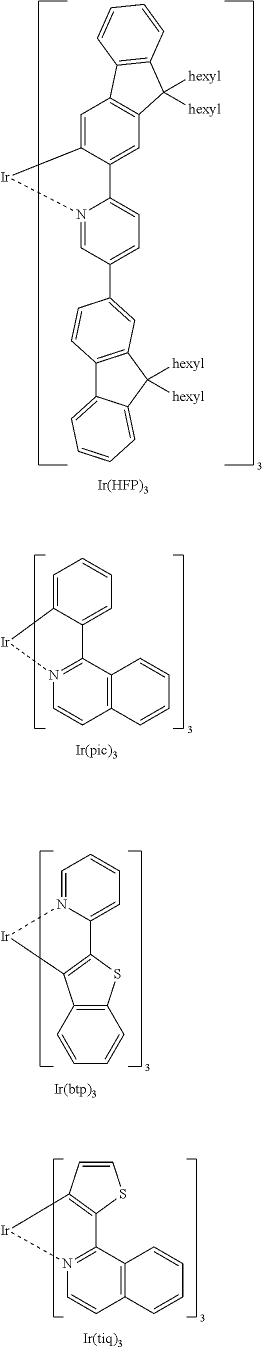

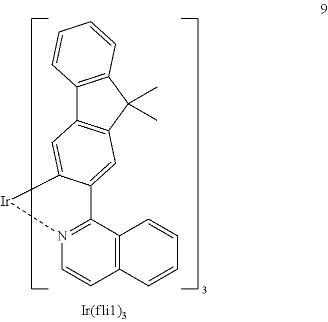

1. (Btp)₂Ir(III)(acac); Bis[2-(2′-benzothienyl)-pyridinato-N,C3′]iridium (III)(acetylacetonate)
2. (Pq)₂Ir(III)(acac); Bis[(2-phenylquinolyl)-N,C2′]iridium (III) (acetylacetonate)
3. (Piq)₂Ir(III)(acac); Bis[(1-phenylisoquinolinato-N,C2′)]iridium (III) (acetylacetonate)
4. (DBQ)₂Ir(acac); Bis[(dibenzo[f, h]quinoxalino-N,C2′)iridium (III)(acetylacetonate)
5. [Ir(HFP)₃], Tris(2,5-bis-2′-(9′,9′-dihexylfluorene)pyridine)iridium (III)
6. Ir(piq)₃; Tris[1-phenylisoquinolinato-N,C2′]iridium (III)
7. Ir(btp)₃; Tris-[2-(2′-benzothienyl)-pyridinato-N,C3′]iridium (III)
8. Ir(tiq)₃, Tris[1-thiophen-2-ylisoquinolinato-N,C3′]iridium (III)
9. Ir(fliq)₃; Tris[1-(9,9-dimethyl-9H-fluoren-2-yl)isoquinolinato-(N,C3′)iridium (III))

The thickness of the light-emitting layer may vary. In some embodiments, the light-emitting layer may have a thickness in the range of from about 5 nm to about 200 nm or about 10 nm to about 150 nm.

Some devices may be configured so that holes can be transferred from the anode to a light-emitting layer and/or so that electrons can be transferred from the cathode to a light-emitting layer.

The compounds and compositions described herein may be useful in an emissive layer without requiring any additional hole-transport or electron-transport materials. Thus, in some embodiments, the light-emitting layer consists essentially of an electroluminescent compound and a compound disclosed herein. In some embodiments, the light-emitting layer consists essentially of a compound disclosed herein. In some embodiments, the light-emitting layer may comprise at least one hole-transport material or electron-transport material in addition to a compound disclosed herein.

A hole-transport layer, e.g. hole-transport layer 15, may be disposed between the anode and the light-emitting layer. The hole-transport layer may comprise at least one hole-transport material. In some embodiments, the hole-transport material comprises at least one of an aromatic-substituted amine, a carbazole, a polyvinylcarbazole (PVK), e.g. poly(9-vinylcarbazole); polyfluorene; a polyfluorene copolymer; poly(9,9-di-n-octylfluorene-alt-benzothiadiazole); poly(paraphenylene); poly[2-(5-cyano-5-methylhexyloxy)-1,4-phenylene]; a benzidine; a phenylenediamine; a phthalocyanine metal complex; a polyacetylene; a polythiophene; a triphenylamine; copper phthalocyanine; 1,1-Bis(4-bis(4-methylphenyl) aminophenyl) cyclohexane; 2,9-Dimethyl-4,7-diphenyl-1,10-phenanthroline; 3,5-Bis(4-tert-butyl-phenyl)-4-phenyl[1,2,4]triazole; 3,4,5-Triphenyl-1,2,3-triazole; 4,4′,4′-tris(3-methylphenylphenylamino)triphenylamine (MTDATA); N,N′-bis(3-methylphenyl)N,N′-diphenyl-[1,1′-biphenyl]-4,4′-diamine (TPD); 4,4′-bis[N-(naphthyl)-N-phenyl-amino]biphenyl (NPB); 4,4′,4″-tris(carbazol-9-yl)-triphenylamine (TCTA); 4,4′-bis[N,N′-(3-tolyl)amino]-3,3′-dimethylbiphenyl (HMTPD); 4,4′-N,N′-dicarbazole-biphenyl (CBP); 1,3-N,N-dicarbazole-benzene (mCP); Bis[4-(p,p′-ditolyl-amino)phenyl]diphenylsilane (DTASi); 2,2′-bis(4-carbazolylphenyl)-1,1′-biphenyl (4CzPBP); N,N′N″-1,3,5-tricarbazoloylbenzene (tCP); N,N′-bis(4-butylphenyl)-N,N′-bis(phenyl)benzidine; or the like.

A hole-injection layer, e.g. hole-injecting layer 10, may be disposed between the light-emitting layer and the anode. Various suitable hole-injection materials that can be included in the hole-injection layer are known to those skilled in the art. Examples of hole-injection material(s) include 1,4,5,8,9,11-hexaazatriphenylene-hexacarbonitrile (HAT-CN), MoO₃, V₂O₅, WO₃, or an optionally substituted compound selected from the following: a polythiophene derivative such as poly(3,4-ethylenedioxythiophene (PEDOT)/polystyrene sulphonic acid (PSS), a benzidine derivative such as N, N, N′, N′-tetraphenylbenzidine, poly(N,N′-bis(4-butylphenyl)-N,N′-bis(phenyl)benzidine), a triphenylamine or phenylenediamine derivative such as N,N′-bis(4-methylphenyl)-N,N′-bis(phenyl)-1,4-phenylenediamine, 4,4′,4″-tris(N-(naphthylen-2-yl)-N-phenylamino)triphenylamine, an oxadiazole derivative such as 1,3-bis(5-(4-diphenylamino)phenyl-1,3,4-oxadiazol-2-yl)benzene, a polyacetylene derivative such as poly(1,2-bis-benzylthio-acetylene), and a phthalocyanine metal complex derivative such as phthalocyanine copper (CuPc). In some embodiments, hole-injection materials, while still being able to transport holes, may have a hole mobility substantially less than the hole mobility of conventional hole transport materials. A p-doped hole injecting layer, e.g. p-doped hole injection layer 12, may include a hole injecting material doped with a hole-transport material, for example a p-doped hole injecting layer may comprise MoO₃ doped with NPB.

An electron-transport layer, e.g. electron-transport layer 30, may be disposed between the cathode and the light-emitting layer. In some embodiments, the electron-transport layer may comprise a compound described herein. Other electron-transport materials may be included, such as 2-(4-biphenylyl)-5-(4-tert-butylphenyl)-1,3,4-oxadiazole (PBD); 1,3-bis(N,N-t-butyl-phenyl)-1,3,4-oxadiazole (OXD-7), 1,3-bis[2-(2,2′-bipyridine-6-yl)-1,3,4-oxadiazo-5-yl]benzene, 3-phenyl-4-(1′-naphthyl)-5-phenyl-1,2,4-triazole (TAZ); 2,9-dimethyl-4,7-diphenyl-phenanthroline (bathocuproine or BOP); aluminum tris(8-hydroxyquinolate) (Alq3); and 1,3,5-tris(2-N-phenylbenzimidazolyl)benzene; 1,3-bis[2-(2,2′-bipyridine-6-yl)-1,3,4-oxadiazo-5-yl]benzene (BPY-OXD); 3-phenyl-4-(1′-naphthyl)-5-phenyl-1,2,4-triazole (TAZ), 2,9-dimethyl-4,7-diphenyl-phenanthroline (bathocuproine or BOP); and 1,3,5-tris[2-N-phenylbenzimidazol-z-yl]benzene (TPBI). In some embodiments, the electron transport layer may be aluminum quinolate (Alq₃), phenanthroline, quinoxaline, 1,3,5-tris[N-phenylbenzimidazol-z-yl] benzene (TPBI), or a derivative or a combination thereof.

In some embodiments, the light-emitting device can include an electron injection layer, e.g. electron injecting layer 25, between the cathode layer and the light-emitting layer. In some embodiments, the lowest unoccupied molecular orbital (LUMO) energy level of the electron injection material(s) may be high enough to prevent it from receiving an electron from the light-emitting layer. In other embodiments, the energy difference between the LUMO of the electron injection material(s) and the work function of the cathode layer is small enough to allow the electron injection layer to efficiently inject electrons into the light-emitting layer from the cathode. A number of suitable electron injection materials are known to those skilled in the art. Examples of suitable electron injection material(s) include but are not limited to, an optionally substituted compound selected from the following: LiF, CsF, Cs doped into electron transport material as described above or a derivative or a combination thereof.

A substrate, e.g. substrate 1, may be any material, such as a glass or a metal, upon which the light-emitting diode may be mounted.

A heat dissipation layer, e.g., the heat dissipation layer 3, includes any layer of material that may be capable of increasing the surface area of the device for thermal exchange, spreading the heat uniformly throughout the device area, transferring the heat to the heat sink materials, and/or releasing the heat outside of the device. A typical heat dissipation layer may include, but is not limited to: an aluminum sheet with a fin structure, aluminum tape with thermal conductive adhesive, a copper thin film, a graphite sheet, a stainless steel film, a Si-wafer, a thin film of boron nitride, a thermal conductive grease, a gel, or combinations of above.

A capping layer, e.g., the capping layer 40, may be any layer that enhances the emission of light from an OLED device. An enhancement layer may comprise any material that is capable of increasing the emission of light by an OLED device. Examples of such materials may include, but are not limited to, transparent materials including organic small molecule materials such as NPB, TPBI, Alq3; metal oxides such as MoO₃, WO₃, SnO₂ and SnO; wide band gap semiconductor compounds; etc. Additional examples include enhancement layers and/or porous films as described in co-pending patent application, entitled, “Formation of high efficient porous nano-structured light outcoupling film for organic light emitting diodes and the use of the same” (Ser. No. 61/449,032, filed 3 Mar. 2011), which is incorporated in its entirety, herein.

If desired, additional layers may be included in the light-emitting device. These additional layers may include a hole-blocking layer (HBL) and/or an exciton-blocking layer (EBL). In addition to separate layers, some of these materials may be combined into a single layer.

If present, a hole-blocking layer may be between a cathode and a light-emitting layer. Various suitable hole-blocking materials that can be included in the hole-blocking layer are known to those skilled in the art. Suitable hole-blocking material(s) include but are not limited to, an optionally substituted compound selected from the following: bathocuproine (BCP), 3,4,5-triphenyl-1,2,4-triazole, 3,5-bis(4-tert-butyl-phenyl)-4-phenyl-[1,2,4] triazole, 2,9-dimethyl-4,7-diphenyl-1,10-phenanthroline, and 1,1-bis(4-bis(4-methylphenyl)aminophenyl)-cyclohexane.

If present, an exciton-blocking layer may be between a light-emitting layer and an anode. In an embodiment, the band gap of the material(s) that comprise an exciton-blocking layer may be large enough to substantially prevent the diffusion of excitons. A number of suitable exciton-blocking materials that can be included in an exciton-blocking layer are known to those skilled in the art. Examples of material(s) that can compose an exciton-blocking layer include an optionally substituted compound selected from the following: aluminum quinolate (Alq₃), 4,4′-bis[N-(naphthyl)-N-phenyl-amino]biphenyl (NPB), 4,4′-N,N′-dicarbazole-biphenyl (CBP), and bathocuproine (BCP), and any other material(s) that have a large enough band gap to substantially prevent the diffusion of excitons.

Light-emitting devices comprising the compounds disclosed herein can be fabricated using techniques known in the art, as informed by the guidance provided herein. For example, a glass substrate can be coated with a high work functioning metal such as ITO which can act as an anode. After patterning the anode layer, a light-emitting layer that includes at least a compound disclosed herein, and an optional electroluminescent compound, can be deposited on the anode. A cathode layer, comprising a low work functioning metal (e.g., Mg:Ag), can then be deposited, e.g., vapor evaporated, onto the light-emitting layer. If desired, the device can also include an electron-transport/injection layer, a hole-blocking layer, a hole-injection layer, an exciton-blocking layer and/or a second light-emitting layer that can be added to the device using techniques known in the art, as informed by the guidance provided herein.

### Phototherapy

Devices disclosed herein may be useful in phototherapy. Typically, phototherapy involves exposing at least a portion of the tissue of a mammal to light, such as light from a device described herein. For example, at least a portion of light emitted from a device described herein may come in contact with a mammal, such as a human being. The light from the device that comes in contact with the mammal may then provide a therapeutic effect to the mammal through a variety of mechanisms, some of which are explored later in this document.

A device may be configured to emit an effective amount of light to provide a therapeutic effect to a mammal. A therapeutic effect includes any detectable, diagnosis, cure, mitigation, treatment, or prevention of disease, or other effect on the structure or function of the body of a mammal. Some examples of conditions that phototherapy may be useful to treat or diagnose include, but are not limited to, infection, cancer/tumors, cardiovascular conditions, dermatological or skin conditions, a condition affecting the eye, obesity, pain or inflammation, conditions related to immune response, etc.

Examples of infections may include microbial infection such as bacterial infection, viral infection, fungal infection, protozoal infection, etc.

Examples of cancer or tumor tissues include vascular endothelial tissue, an abnormal vascular wall of a tumor, a solid tumor, a tumor of a head, a tumor of the brain, a tumor of a neck, a tumor of a gastrointestinal tract, a tumor of a liver, a tumor of a breast, a tumor of a prostate, a tumor of a lung, a nonsolid tumor, malignant cells of one of a hematopoietic tissue and a lymphoid tissue, lesions in a vascular system, a diseased bone marrow, diseased cells in which the disease may be one of an autoimmune and an inflammatory disease, etc.

Examples of cardiovascular conditions may include myocardial infarction, stroke, lesions in a vascular system, such as atherosclerotic lesions, arteriovenous malformations, aneurysms, venous lesions, etc. For example, a target vascular tissue may be destroyed by cutting off circulation to the desired location.

Examples of dermatological or skin conditions may include hair loss, hair growth, acne, psoriasis, wrinkles, discoloration, skin cancer, rosacea, etc.

Examples of eye conditions may include age related macular degeneration (AMD), glaucoma, diabetic retinopathy, neovascular disease, pathological myopia, ocular histoplasmosis, etc.

Examples of pain or inflammation include arthritis, carpal tunnel, metatarsalgia, plantar fasciitis, TMJ, pain or inflammation affecting an elbow, an ankle, a hip, a hand, etc. Examples of conditions related to immune response may include HIV or other autoimmune disease, organ transplant rejection, etc.

Other non-limiting uses of phototherapy may include treating benign prostate hyperplasia, treating conditions affecting adipose tissue, wound healing, inhibiting cell growth, and preserving donated blood.

The color of the light used for phototherapy may vary according to the particular treatment, the absorption spectrum of any photosensitive compounds, and other factors. For example, light in the red to ultraviolet range may be useful for penetrating tissue. In some embodiments, an OLED used in phototherapy may have peak emission or an average emission (e.g. the wavelength having equal areas in the visible spectra higher and lower than the wavelength) at a wavelength of about 600 nm to about 800 nm, about 600 nm to about 700 nm, about 630 nm to about 700 nm, about 609 nm, about 630 nm, about 635 nm, about 652 nm, about 660 nm, about 664 nm, about 668 nm, about 693 nm, about 732 nm, about 765 nm, about 800 nm, or any wavelength in a range bounded by, or between, any of these values.

The light itself may be at least partially responsible for the therapeutic effects of the phototherapy, thus phototherapy may be carried out without a photosensitive compound. In embodiments where a photosensitive compound is not used, light in the red range (approximately 630 nm to 700 nm) may decrease inflammation in injured tissue, increase ATP production, and otherwise stimulate beneficial cellular activity.

In some embodiments, light in the red range (approximately 600 nm to 700 nm) can be used in combination with wound dressings to effect accelerated wound healing. The wound dressing may include a hydrocolloid particles or material, for example as described in US 20080311178 (Ishikura, Jun, et al, filed Jun. 4, 2008); a transparent film, for example as described in U.S. Pat. No. 7,678,959 issued Mar. 16, 2010 to Okadam Katshiro, et al.; and/or an adhesive material. An adhesive may be any conventional adhesive and may have sufficient adhesion to keep the wound dressing or device in contact with a patient while not having too much adhesion such that wound dressing cannot be removed from the patient. In these methods, a wound dressing may be used alone or in combination with a photosensitive compound.

In some embodiments, at least a portion of a wound dressing is exposed to light from a device. The wound dressing may be applied to the wound of a mammal to effect accelerated healing. The dressing may be exposed to the light prior to and/or subsequent to application of the dressing to the wound site. Light in the red range may also be used in conjunction with light of other spectral wavelengths, for example blue or yellow, to facilitate post operative healing.

Facial rejuvenation may be effected by applying light having a wavelength of about 633 nm to the desired tissue for about 20 minutes. In some embodiments, facial skin rejuvenation is believed to be attained by applying light in the red range for a therapeutically effective amount of time.

The light may also be used in conjunction with a photosensitive compound. The photosensitive compound may be administered directly or indirectly to body tissue so that the photosensitive compound is in or on the tissue. Since the photosensitive compound is in or on the tissue, at least a portion of the photosensitive compound may be exposed to light emitted from the device and directed toward or through the tissue. The photosensitive compound may thus be activated by light from the device.

Activation of a compound may change the compound in such a way that the compound or a reaction product of the activated compound may have therapeutic effect in vivo. For example, a compound may be activated by absorbing light to transition to an excited electronic state, such as an excited singlet or triplet state. A compound in an excited electronic state may then react to form physiologically active compounds. A compound in an excited electronic state may also directly or indirectly form reactive species such as radicals (including singlet oxygen radicals), radical ions, carbenes, or the like, which can readily react with materials in living cells or tissue.

For example, a photosensitive compound may be administered systemically by ingestion or injection, topically applying the compound to a specific treatment site on a patient's body, or by some other method. This may be followed by illumination of the treatment site with light having a wavelength or waveband corresponding to a characteristic absorption waveband of the photosensitive compound, such as at least about 500 or about 600 nm; and/or up to about 800 nm or about 1100 nm. Illumination in this manner may activate the photosensitive compound. Activating the photosensitive compound may cause singlet oxygen radicals and other reactive species to be generated, which may lead to a number of biological effects that may destroy the tissue which has absorbed the photosensitive compound such as abnormal or diseased tissue.

A photosensitive compound may be any compound, or pharmaceutically acceptable salts, prodrugs, or hydrates thereof, which may react as a direct or indirect result of absorption of ultraviolet, visible, or infrared light. In one embodiment, the photosensitive compound may react as a direct or indirect result of absorption of red light. The photosensitive compound may be a compound which is not naturally in the tissue. Alternatively, the photosensitive compound may naturally be present in the tissue, but an additional amount of the photosensitive compound may be administered to the mammal. In some embodiments, the photosensitive compound may selectively bind to one or more types of selected target cells and, when exposed to light of an appropriate waveband, may absorb the light, which may cause substances to be produced that impair or destroy the target cells.

While not limiting any embodiment, for some types of therapies, it may be helpful if the photosensitive compound has sufficiently low toxicity, or can be formulated to have sufficiently low toxicity, so as not to cause more harm than the disease or the condition that is to be treated with the phototherapy. In some embodiments, it may also be helpful if the photodegradation products of the photosensitive compounds are nontoxic.

Some non-limiting examples of photosensitive compounds or materials may be found in Kreimer-Bimbaum, Sem. Hematol, 26:157-73, (1989), incorporated by reference herein in its entirety, and may include, but are not limited to, chlorins, e.g., Tetrahydroxylphenyl chlorin (THPC) [652 nm], bacteriochlorins [765 nm], e.g., N-Aspartyl chlorin e6 [664 nm], phthalocyanines [600-700 nm], porphyrins, e.g., hematoporphyrin [HPD] [630 nm], purpurins, e.g., [1,2,4-Trihydroxyanthraquinone] Tin Etiopurpurin [660 nm], merocyanines, psoralens, benzoporphyrin derivatives (BPD), e.g., verteporfin, and porfimer sodium; and pro-drugs such as delta-aminolevulinic acid or methyl aminolevulinate, which can produce photosensitive agents such as protoporphyrin IX. Other suitable photosensitive compounds may include indocyanine green (ICG) [800 nm], methylene blue [668 nm, 609 nm], toluidine blue, texaphyrins, Talaportin Sodium (mono-L-aspartyl chlorine)[664 nm], verteprofin [693 nm], which may be useful for phototherapy treatment of conditions such as age-related macular degeneration, ocular histoplasmosis, or pathologic myopia], lutetium texaphyrin [732 nm], and rostaporfin [664 nm].

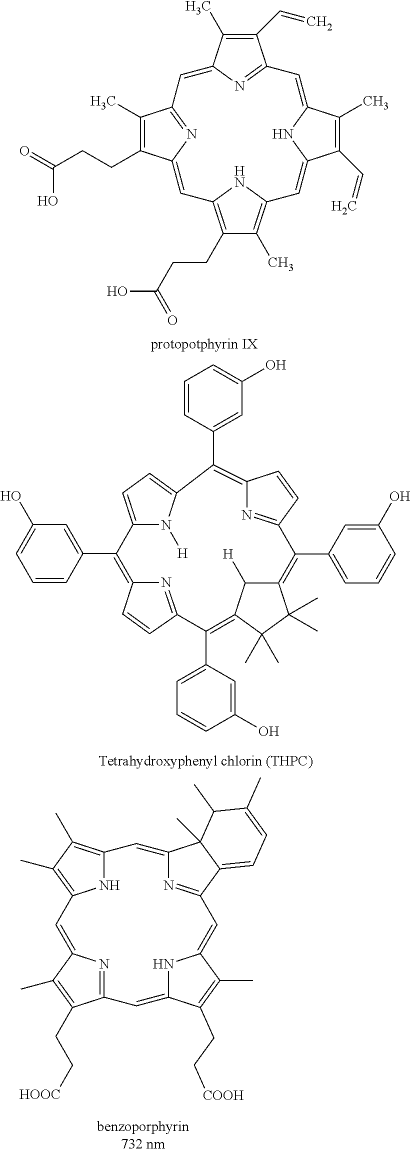

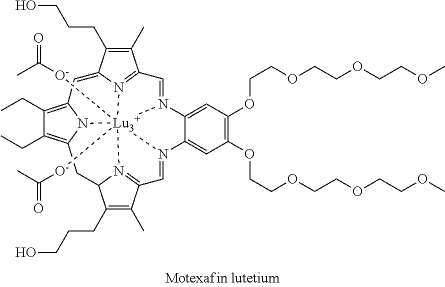

In some embodiments, the photosensitive compound comprises at least one component of porfimer sodium. Porfimer sodium comprises a mixture of oligomers formed by ether and ester linkages of up to eight porphorin units. The structural formula below is representative of some of the compounds present in porfimer sodium, wherein n may be 0, 1, 2, 3, 4, 5, or 6 and each R may be independently —CH(OH)CH₃ or —CH═CH₂.

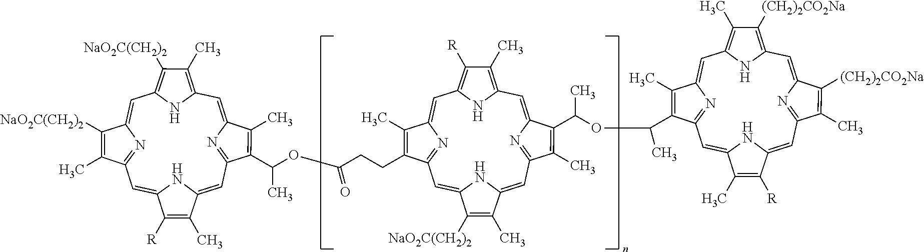

In some embodiments, the photosensitive compound may be at least one of the regioisomers of verteporphin, shown below.

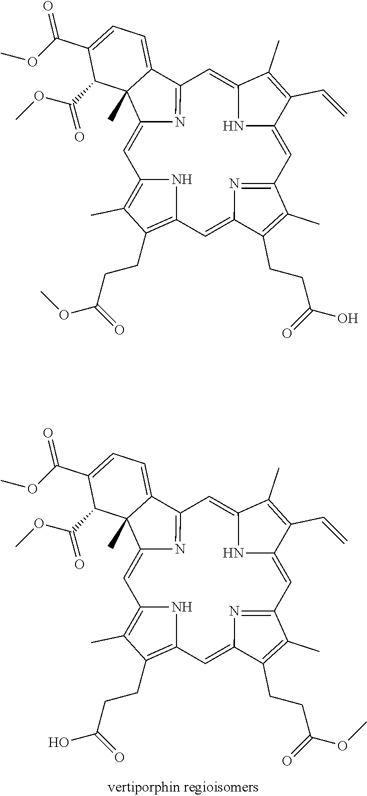

In some embodiments, the photosensitive compound may comprise a metal analogue of phthalocyanine shown below.

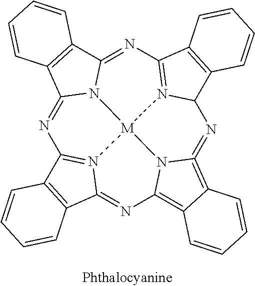

In one embodiment, M may be zinc. In one embodiment, the compound can be zinc phthalocyanine or zinc phthalocyanine tetrasulfonate.

In some embodiments, a photosensitive agent may be 5-aminolevulinic acid, methyl aminolevulinate, verteporfin, zinc phthalocyanine, or a pharmaceutically acceptable salt thereof. In some embodiments, the photosensitive compound is 5-aminolevulinic acid.

A photosensitive agent can be administered in a dry formulation, such as a pill, a capsule, a suppository or a patch. The photosensitive agent may also be administered in a liquid formulation, either alone, with water, or with pharmaceutically acceptable excipients, such as those disclosed in Remington's Pharmaceutical Sciences. The liquid formulation also can be a suspension or an emulsion. Liposomal or lipophilic formulations may be desirable. If suspensions or emulsions are utilized, suitable excipients may include water, saline, dextrose, glycerol, and the like. These compositions may contain minor amounts of nontoxic auxiliary substances such as wetting or emulsifying agents, antioxidants, pH buffering agents, and the like. The above described formulations may be administered by methods which may include but are not limited to intradermal, intramuscular, intraperitoneal, intravenous, subcutaneous, intranasal, epidural, oral, sublingual, intranasal, intracerebral, intravaginal, transdermal, iontophoretical, rectally, by inhalation, or topically to the desired target area, for example, the body cavity (e.g. oral, nasal, rectal), ears, nose, eyes, or skin. The preferred mode of administration may be left to the discretion of the practitioner, and may depend in part upon the site of the medical condition (such as the site of cancer or viral infection).

The dose of photosensitive agent may vary. For example, the target tissue, cells, or composition, the optimal blood level, the animal's weight, and the timing and duration of the radiation administered, may affect the amount of photosensitive agent used. Depending on the photosensitive agent used, an equivalent optimal therapeutic level may be empirically established. The dose may be calculated to obtain a desired blood level of the photosensitive agent, which in some embodiments may be from about 0.001 g/mL or 0.01 μg/ml to about 100 μg/ml or about 1000 μg/ml.

In some embodiments, 5-aminolevulinic acid may be present in tissue at a concentration of about 0.5 to about 2 mM.

In some embodiments, about 0.05 mg/kg or about 1 mg/kg to about 50 mg/kg or about 100 mg/kg of a photosensitive agent such as 5-aminolevulinic acid may be administered to the mammal. Alternatively, for topical application, about 0.15 mg/m² or about 5 mg/m² to about 30 mg/m² or about 50 mg/m² may be administered to the surface of the tissue.

The light may be administered by an external or an internal light source, such as a light-emitting device (e.g., OLED) as described herein. The intensity of radiation or light used to treat the target cell or target tissue may vary. In some embodiments, the intensity may be in the range of about 0.1 mW/cm² to about 100 mW/cm², about 1 mW/cm² to about 50 mW/cm², or about 3 mW/cm² to about 30 mW/cm². The duration of radiation or light exposure administered to a subject may vary. In some embodiments the exposure ranges from at least about 1 minute, at least about 60 minutes, or at least about 2 hours; and/or up to about 24 hours, about 48 hours, or about 72 hours.

A certain amount of light energy may be required to provide a therapeutic effect. For example, a certain amount of light energy may be required to activate the photosensitive compounds. This may be accomplished by using a higher power light source, which may provide the needed energy in a shorter period of time, or a lower power light source may be used for a longer period of time. Thus, a longer exposure to the light may allow a lower power light source to be used, while a higher power light source may allow the treatment to be done in a shorter time. In some embodiments, the total fluence or light energy administered during a treatment may be in the range of about 5 Joules to about 1,000 Joules, about 20 Joules to about 750 Joules, or about 50 Joules to about 500 Joules. In some embodiments, the light energy administered during a treatment may depend upon the amount of tissue exposed to the light energy. For example, the light dose may be in the range of about 5 Joules/cm² to about 1,000 Joules/cm², about 20 Joules/cm² to about 750 Joules/cm², about 30 Joules/cm² to about 1,000 Joules/cm², about 30 Joules/cm² to about 60 Joules/cm², 50 Joules/cm² to 500 Joules/cm²; or may be about 5 Joules/cm², about 15 Joules/cm², about 20 Joules/cm², about 30 Joules/cm², about 45 Joules/cm², about 50 Joules/cm², about 60 Joules/cm², about 500 Joules/cm², about 750 Joules/cm², about 1,000 Joules/cm², or any light dose in a range bounded by, or between, any of these values.

The intensity of light decreases with the square of the distance from the source of the light. For example, light 1 meter away from a source is four times as intense as light 2 meters from the same source. A dose of light and other properties related to intensity can similarly vary. Thus, unless otherwise stated, distance-dependent properties of light refer to the property at the location of the tissue being treated.

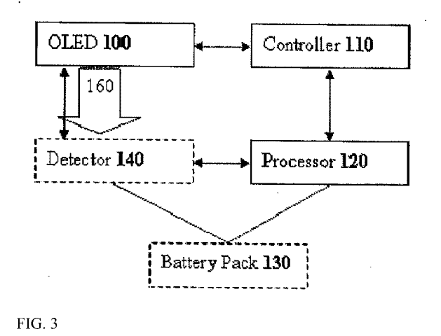

FIG. 3 is a schematic of some embodiments which further include a controller 110 and processor 120 electrically connected to an organic light-emitting diode 100 (OLED), which may help to provide a uniform power supply to facilitate homogeneous light exposure of the tissue. In some embodiments, the apparatus may further include an optional detector 140, such as photodiode, which may detect a portion of the light 160 emitted from the OLED 100, to help determine the amount of light being emitted by the OLED 100. For example, the detector 140 may communicate a signal related to the intensity of the light 160 received from the OLED 100 to the processor 120, which, based upon the signal received, may communicate any desired power output information to the controller 100. Thus, these embodiments may provide real time feedback which allows the control of the intensity of light emitted from the OLED 100. The detector 140 and the processor 120 may be powered by compact power supply, such as a battery pack 130, or by some other power source.

In some embodiments related to phototherapy, the LED device may further comprise a dosage component. A dosage component may be configured to control the device to provide a sufficient amount of light to achieve a therapeutic effect in a person or animal, e.g., a mammal. If a photosensitive compound is used, a dosage component may be configured to control the device to provide a sufficient amount of light to activate a sufficient portion of a photosensitive compound to provide a therapeutic effect for treating a disease in a mammal such as a human being.

For example, a dosage component may comprise a timer that is configured to control delivery of light from the device for an amount of time sufficient to deliver the appropriate light dosage. The timer may automatically stop the emission from the device once the appropriate light dosage has been delivered. The dosage component may also comprise a positioning component that positions the device so that emitted light is delivered to the appropriate area of a mammal body and is at an appropriate distance from the affected tissue to deliver an effective amount of light. The dosage component may be configured to work with a particular photosensitive compound, or may provide flexibility. For example, a physician, a veterinarian, or another appropriate medical practitioner may set the parameters of the dosage component for use by a patient outside of the practitioner's office, such as at the patient's home. In some embodiments, the device may be provided with a set of parameters for various photosensitive compounds to assist a medical practitioner in configuring the device.

In some embodiments, the device may further include a wireless transmitter electrically connected to a component of the apparatus generating treatment information, e.g., level of intensity, time of application, dosage amount, to communicate/transfer data to another external receiving device, like cell phone, PDA or to doctor's office. In some embodiments, the apparatus may further include an adhesive tape which may be used to attach the apparatus on the tissue surface so as to stabilize it on the target area.

For phototherapy and other applications, a wavelength convertor may be positioned in the device to receive at least a portion of light emitted from the organic light-emitting diode in a lower wavelength range, such as about 350 nm to less than about 600 nm, and convert at least a portion of the light received to light in a higher wavelength range, such as about 600 nm to about 800 nm. The wavelength convertor may be a powder, a film, a plate, or in some other form and, may comprise: yttrium aluminum garnet (YAG), alumina (Al₂O₃), yttria (Y₂O₃), titania (TiO₂), and the like. In some embodiments, the wavelength convertor may comprise at least one dopant which is an atom or an ion of an element such as Cr, Ce, Gd, La, Tb, Pr, Sm, Eu, etc.

In some embodiments, translucent ceramic phosphor may be represented by a formula such as, but not limited to (A₁₋ₓEₓ)₃D₅O₁₂, (Y₁₋ₓEₓ)₃D₅O₁₂, (Gd₁₋ₓEₓ)₃D₅O₁₂, (La₁₋ₓEₓ)₃D₅O₁₂, (Lu₁₋ₓEₓ)₃D₅O₁₂, (Tb₁₋ₓEₓ)₃D₅O₁₂, (A₁₋ₓEₓ)₃Al₅O₁₂, (A₁₋ₓEₓ)₃Ga₅O₁₂, (A₁₋ₓEₓ)₃In₅O₁₂, (A₁₋ₓCeₓ)₃D₅O₁₂, (A₁₋ₓEuₓ)₃D₅O₁₂, (A₁₋ₓTbₓ)₃D₅O₁₂, (A₁₋ₓEₓ)₃Nd₅O₁₂, and the like. In some embodiments, the ceramic may comprise a garnet, such as a yttrium aluminum garnet, with a dopant. Some embodiments provide a composition represented by the formula (Y₁₋ₓCeₓ)₃Al₅O₁₂. In any of the above formulas, A may be Y, Gd, La, Lu, Tb, or a combination thereof; D may be Al, Ga, In, or a combination thereof; E may be Ce, Eu, Tb, Nd, or a combination thereof; and x may be in the range of about 0.0001 to about 0.1, from about 0.0001 to about 0.05, or alternatively, from about 0.01 to about 0.03.

According to some embodiments of the present disclosure, the output of light from a phototherapy device is delivered in a non-pulsed manner. In some embodiments, the light is delivered in a pulsed manner and an electronic control circuit or microprocessor is provided to control this pulsing and/or other aspects of device function such as duration of exposure(s) of the area to be treated and the intensity of emitted light.

The output of the phototherapy device may take the form of a train of pulses. In some embodiments, the duration of the pulses is substantially the same as the interval between successive pulses. The period of the pulse train may, for example, be in the range of 20 ms to 2000 s, depending, for example, on the reactivity of the photosensitive compound, if such is used. In some embodiments, the duration between successive pulses is less than or greater than the duration of the pulses. In some embodiments, the duration of the pulses and the duration between pulses is not consistent. For example, the duration of the pulses in some embodiments increases or decreases over time while the duration between pulses remains constant or is varied as well. In some embodiments, the duration between pulses increases or decreases over time as the duration of the pulses is held constant or is varied.

In some embodiments, the duration of a pulse is between about 10 ms and about 3000 s, between about 30 ms and about 1000 s, between about 100 ms and about 100 s, or between about 10 ms and about 1 s.

### SYNTHETIC EXAMPLES

The following are examples of some methods that may be used to prepare compounds described herein.

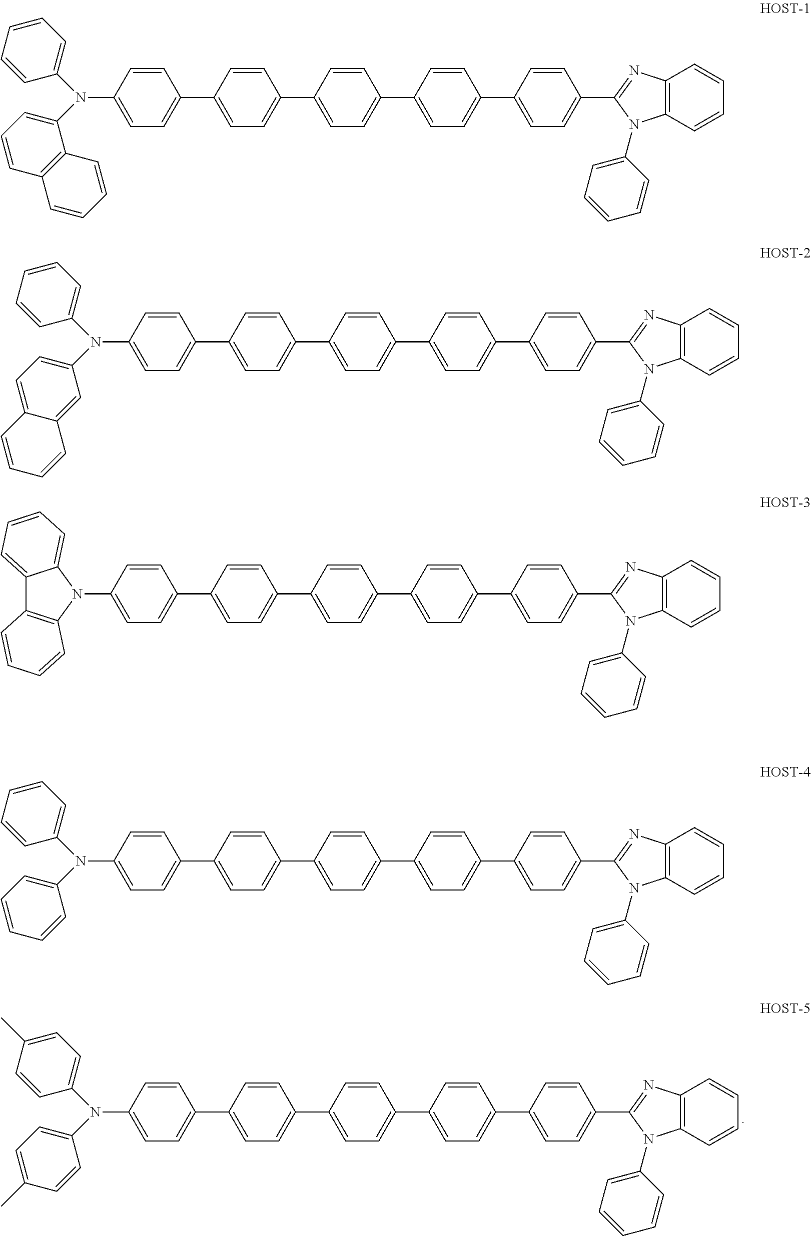

### Synthetic Examples

### Example 1.1

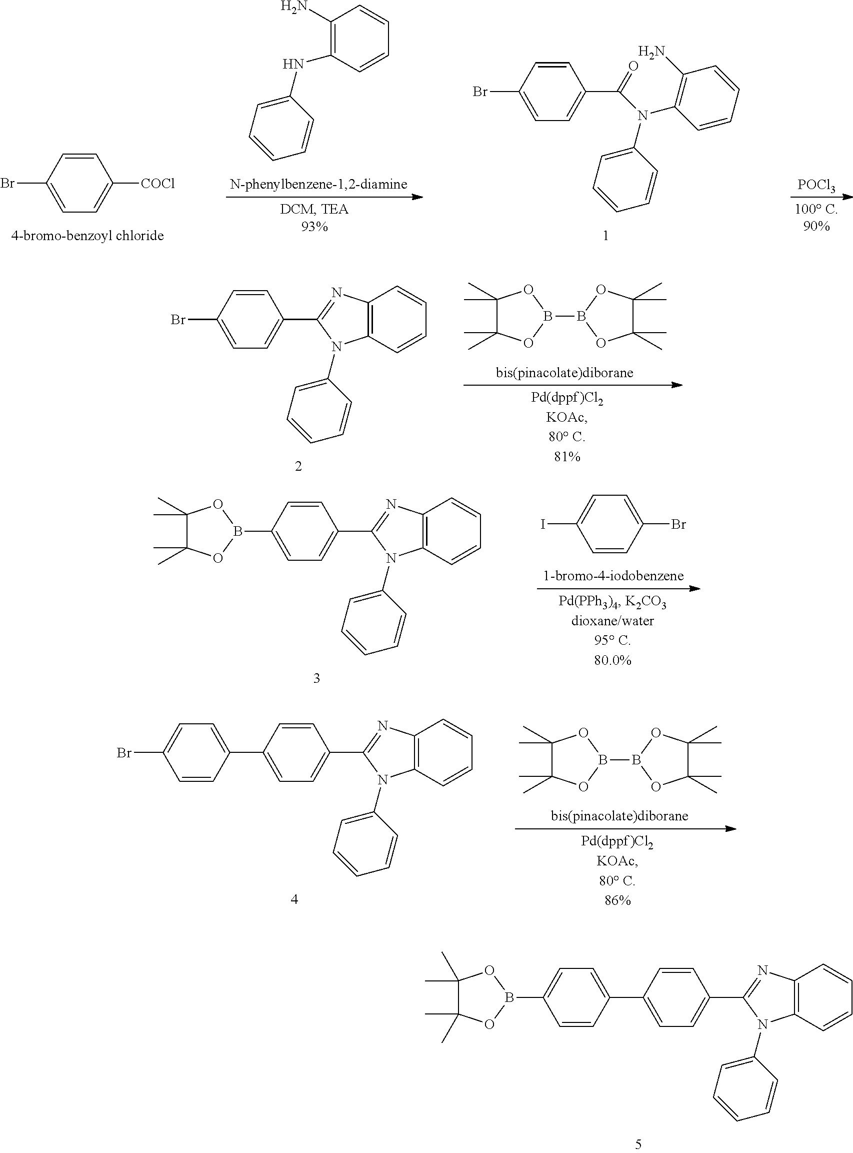

### Example 1.1.1

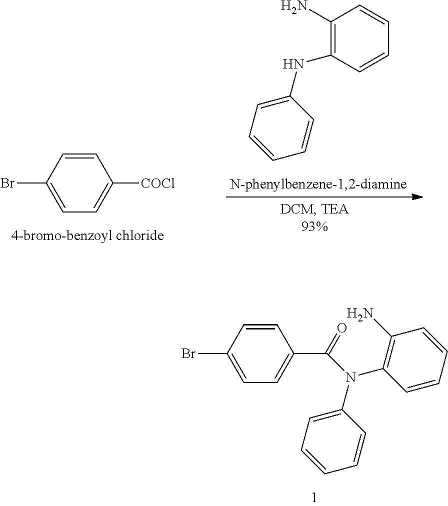 [SMILES: O=C(Cl)c1ccc(Br)cc1 ; Nc1ccccc1Nc1ccccc1 ; Nc1ccccc1N(C(=O)c1ccc(Br)cc1)c1ccccc1]

### 4-Bromo-N-(2-(phenylamino)phenyl)benzamide (1)

To a solution of 4-bromo-benzoyl chloride (11 g, 50 mmol) in anhydrous dichloromethane (DCM) (100 ml), was added N-phenylbenzene-1,2-diamine (10.2 g, 55 mmol), then triethylamine (TEA) (17 ml, 122 mmol) slowly. The whole was stirred at room temperature (RT) overnight. Filtration gave a white solid 1 (6.5 g). The filtrate was worked up with water (300 ml), then extracted with DCM (300 ml) three times. The organic phase was collected and dried over MgSO₄, concentrated and recrystallized in DCM/hexanes to give another portion of white solid 1 (10.6 g). Total amount of product 1 is 17.1 g, in 93% yield.

### Example 1.1.2

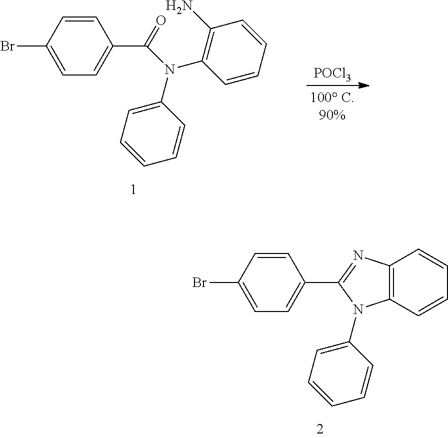

### 2-(4-bromophenyl)-1-phenyl-1H-benzo[d]imidazole (2)

To a suspension of amide 1 (9.6 g, 26 mmol) in anhydrous 1,4-dioxane (100 mL) was added phosphorus oxychloride (POCl₃) (9.2 mL, 100 mmol) slowly. The whole was then heated at 100° C. overnight. After cooling to RT, the mixture was poured into ice (200 g) with stirring. Filtration, followed by recrystallization in DCM/hexanes gave a pale grey solid 2 (8.2 g, in 90% yield).

### Example 1.1.3

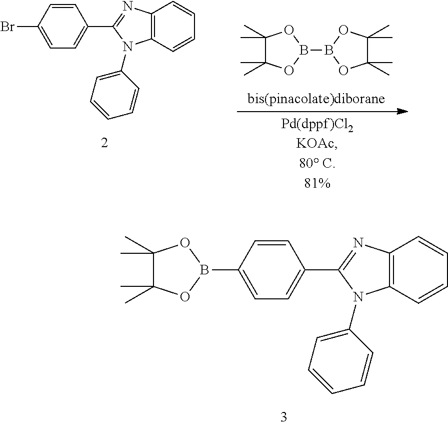

### 1-phenyl-2-(4-(4,4,5,5-tetramethyl-1,3,2-dioxaborolan-2-yl)phenyl)-1H-benzo[d]imidazole (3)

A mixture of Compound 2 (0.70 g, 2 mmol), bis(pinacolate)diborane (0.533 g, 2.1 mmol), bis(diphenylphosphino)ferrocene]dichloropalladium (Pd(dppf)Cl₂) (0.060 g, 0.08 mmol) and anhydrous potassium acetate (KOAc) (0.393 g, 4 mmol) in 1,4-dioxane (20 ml) was heated at 80° C. under argon overnight. After cooling to RT, the whole was diluted with ethyl acetate (80 ml) then filtered. The solution was absorbed on silica gel, then purified by column chromatography (hexanes/ethyl acetate 5:1 to 3:1) to give a white solid 3 (0.64 g, in 81% yield).

### Example 1.1.3

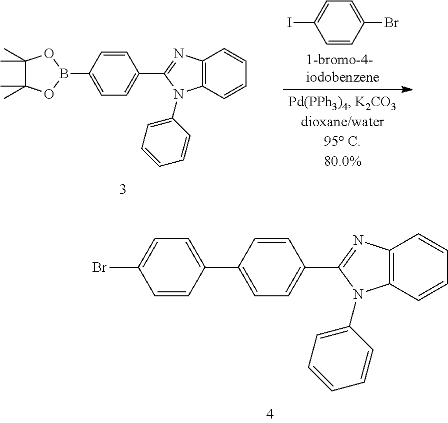 [SMILES: Brc1ccc(I)cc1 ; CC1(C)OB(c2ccc(-c3nc4ccccc4n3-c3ccccc3)cc2)OC1(C)C ; Brc1ccc(-c2ccc(-c3nc4ccccc4n3-c3ccccc3)cc2)cc1]

### 2-(4′-bromo-[1,1′-biphenyl]-4-yl)-1-phenyl-1H-benzo[d]imidazole (4)

A mixture of compound 3 (4.01 g, 10.1 mmol), 1-bromo-4-iodobenzene (5.73 g, 20.2 mmol), Pd(PPh₃)₄ (0.58 g, 0.5 mmol) and potassium carbonate (4.2 g, 30 mmol) in dioxane/water (60 ml/10 ml) was degassed and heated at 95° C. overnight. After being cooled to RT, the mixture was poured into ethyl acetate (250 ml), washed with brine, dried over Na₂SO₄, then loaded on silica gel, purified by flash column (hexanes to hexanes/ethyl acetate 4:1) to give a light yellow solid washed with methanol and dried in air (3.39 g, in 80% yield).

### Example 1.1.4

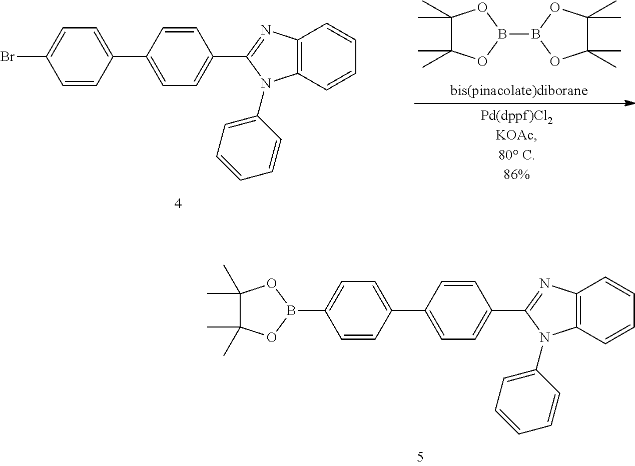

1-phenyl-2-(4′-(4,4,5,5-tetramethyl-1,3,2-dioxaborolan-2-yl)-[1,1′-biphenyl]-4-yl)-1H-benzo[d]imidazole (5): A mixture of Compound 4 (1.2 g, 2.82 mmol), bis(pinacolate)diborane (0.72 g, 2.82 mmol), bis(diphenylphosphino)ferrocene]dichloropalladium (Pd(dppf)Cl₂) (0.10 g, 0.14 mmol) and anhydrous potassium acetate (KOAc) (2.0 g, 20 mmol) in 1,4-dioxane (45 ml) was heated at 80° C. under argon overnight. After cooling to RT, the whole was diluted with ethyl acetate (150 ml) then filtered. The solution was absorbed on silica gel, then purified by column chromatography (hexanes/ethyl acetate 5:1 to 3:1) to give a white solid 5 (1.14 g, in 86% yield).

### Example 1.2

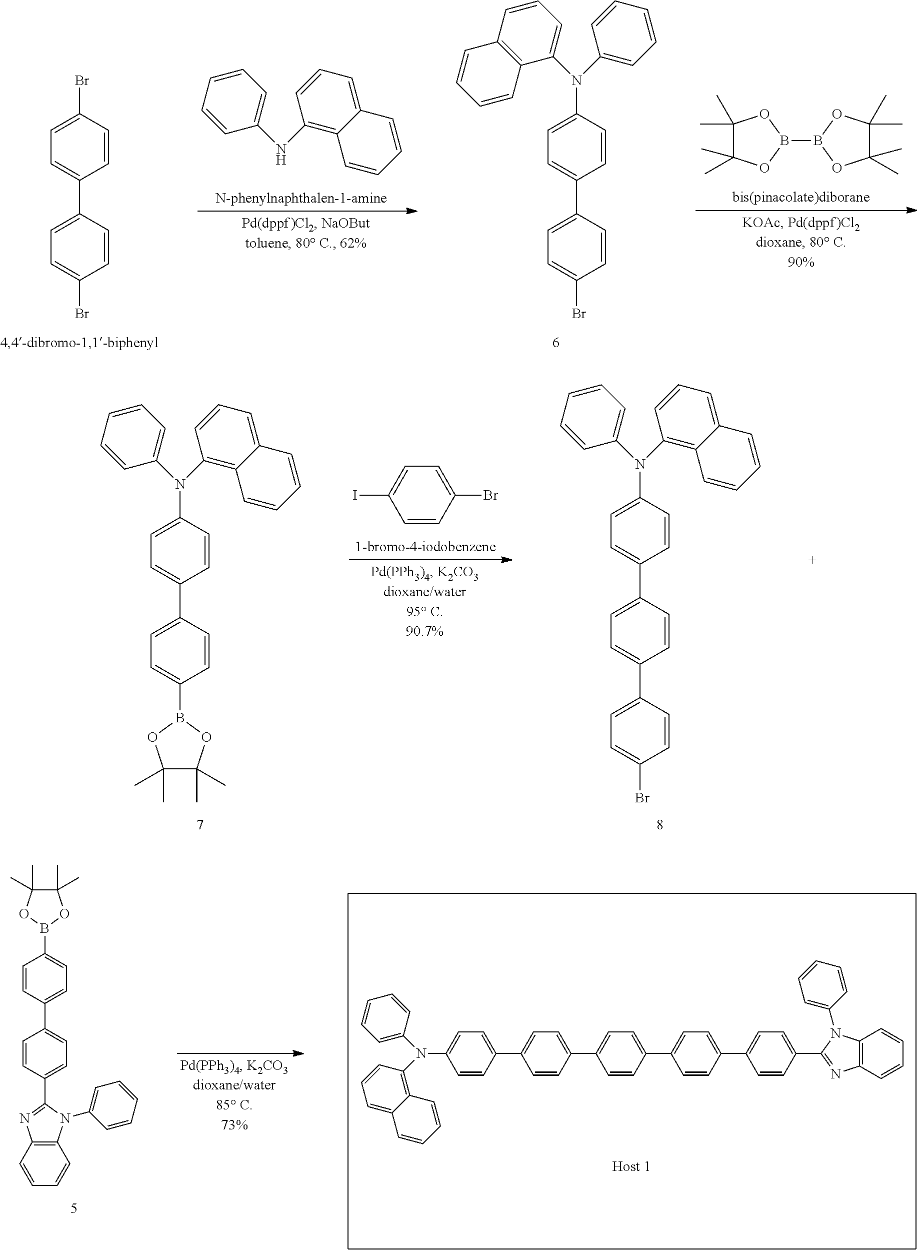

### Example 1.2.1

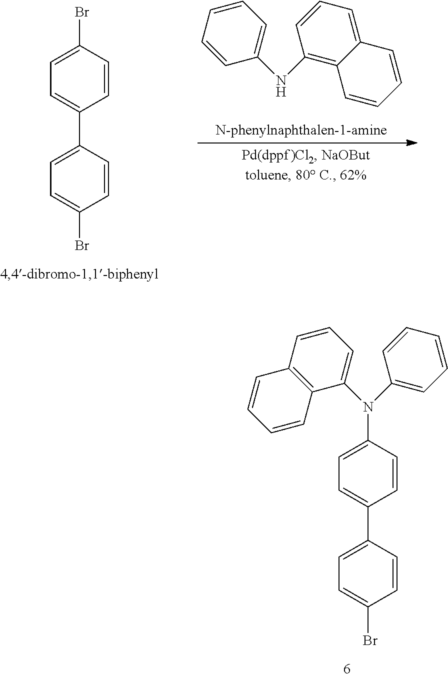 [SMILES: Brc1ccc(-c2ccc(Br)cc2)cc1 ; c1ccc(Nc2cccc3ccccc23)cc1 ; Brc1ccc(-c2ccc(N(c3ccccc3)c3cccc4ccccc34)cc2)cc1]

### N-(4′-bromo-[1,1′-biphenyl]-4-yl)-N-phenylnaphthalen-1-amine (6)

A mixture of N-phenylnaphthalen-1-amine (4.41 g, 20 mmol), 4,4′-dibromo-1,1′-biphenyl (15 g, 48 mmol), sodium tert-butoxide (4.8 g, 50 mmol) and Pd(dppf)Cl₂ (0.44 g, 0.6 mmol) in anhydrous toluene (100 ml) was degassed and heated at 80° C. for 10 hours. After cooling to RT, the mixture was poured into dichloromethane (400 ml) and stirred for 30 min, then washed with brine (100 ml). The organic is collected and dried over Na₂SO₄, loaded on silica gel, and purified by flash column (hexanes to hexanes/ethyl acetate 90:1) to give a solid which was washed with methanol and dried under air to give a white solid 4 (5.58 g, in 62% yield).

### Example 1.2.2

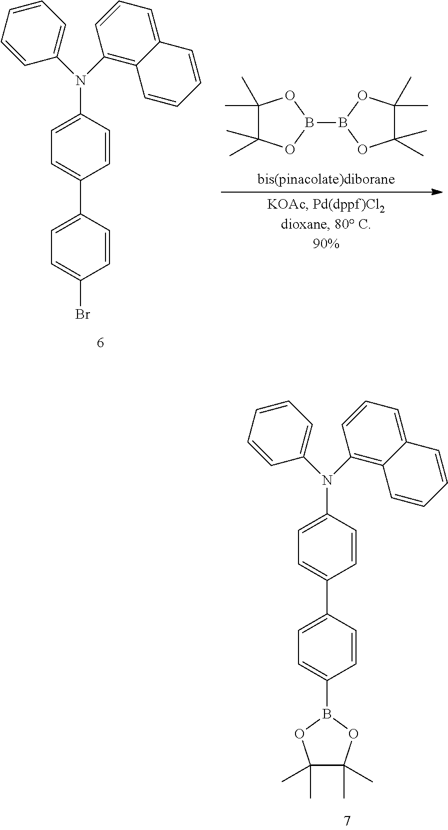

### N-phenyl-N-(4′-(4,4,5,5-tetramethyl-1,3,2-dioxaborolan-2-yl)-[1,1′-biphenyl]-4-yl)naphthalen-1-amine (7)

A mixture of Compound 6 (5.5 g, 12.2 mmol), bis(pinacolate)diborane (3.10 g, 12.2 mmol), Pd(dppf)Cl₂ (0.446 mg, 0.6 mmol) and KOAc (5.5 g, 56 mmol) in anhydrous dioxane (60 ml) was degassed and heated at 80° C. overnight. After being cooled to RT, the mixture was poured into ethyl acetate (200 ml), and washed with brine (150 ml). The organic solution was dried over Na₂SO₄, loaded on silica gel and purified by flash column (hexanes to hexanes/ethyl acetate 30:1) to collect the major fraction. After removal of solvent, the solid was washed with methanol, filtered and dried in air to give a white solid 7 (5.50 g, in 90% yield).

### Example 1.2.3

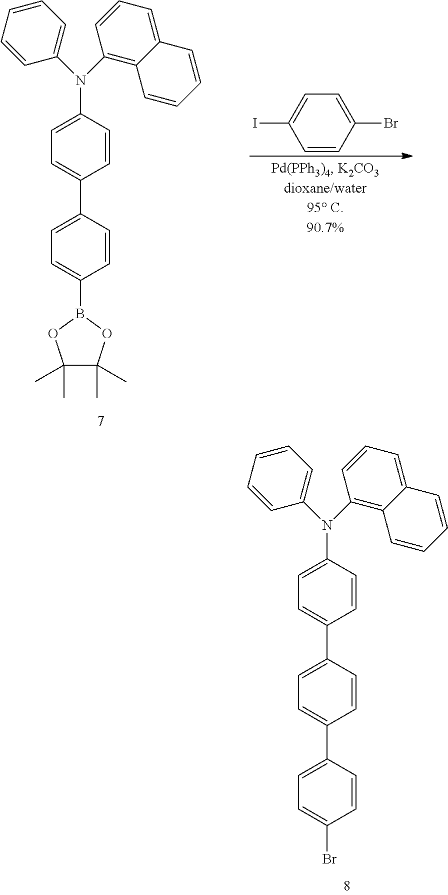 [SMILES: CC1(C)OB(c2ccc(-c3ccc(N(c4ccccc4)c4cccc5ccccc45)cc3)cc2)OC1(C)C ; Brc1ccc(I)cc1 ; Brc1ccc(-c2ccc(-c3ccc(N(c4ccccc4)c4cccc5ccccc45)cc3)cc2)cc1]

### N-(4″-bromo-[1,1′:4′,1″-terphenyl]-4-yl)-N-phenylnaphthalen-1-amine (8)

A mixture of compound 7 (4.5 g, 9.0 mmol), 1-bromo-4-iodobenzene (5.12 g, 18 mmol), Pd(PPh₃)₄ (0.52 g, 0.45 mmol) and potassium carbonate (4.436 g, 32 mmol) in dioxane/water (150 ml/30 ml) was degassed and heated at 95° C. overnight. After being cooled to RT, the mixture was poured into dichloromethane (300 ml), washed with brine, dried over Na₂SO₄, then loaded on silica gel, purified by flash column (hexanes to hexanes/ethyl acetate 20:1) to give a light yellow solid 8 (4.30 g, in 90.7 yield).

### Example 1.2.4

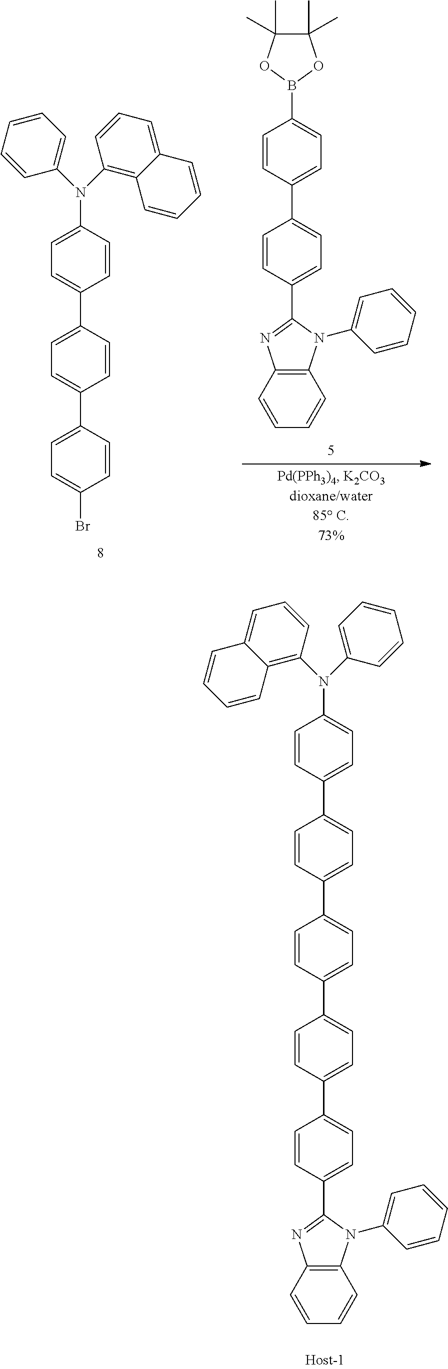 [SMILES: Brc1ccc(-c2ccc(-c3ccc(N(c4ccccc4)c4cccc5ccccc45)cc3)cc2)cc1 ; CC1(C)OB(c2ccc(-c3ccc(-c4nc5ccccc5n4-c4ccccc4)cc3)cc2)OC1(C)C ; c1ccc(N(c2ccc(-c3ccc(-c4ccc(-c5ccc(-c6ccc(-c7nc8ccccc8n7-c7ccccc7)cc6)cc5)cc4)cc3)cc2)c2cccc3ccccc23)cc1]

Host-1:

A mixture of compound 8 (1.50 g, 2.47 mmol), compound 5 (1.11 g, 2.35 mmol), Pd(PPh₃)₄ (0.16 g, 0.14 mmol) and potassium carbonate (1.38 g, 10 mmol) in dioxane/water (60 ml/10 ml) was degassed and heated at 85° C. for 18 hours. After being cooled to RT, the mixture was filtered. The solid and the filtrate were collected separately. The solid from the first filtration was redissolved in dichloromethane (100 ml), loaded on silica gel, and purified by flash column (dichloromethane to dichloromethane/ethyl acetate 9:1) to collect the desired fraction, concentrated. The white precipitate was filtered and dried in air to give a light yellow solid, Host-1 (1.35 g). The overall yield is 73%. LCMS data: calcd for C₅₉H₄₂N₃ (M+H): 792.3. found m/e=792.

### Example 2

### OLED Device Configuration and Performance

A device configured as shown in FIG. 1 may be prepared as described below. Such a device comprises the following layers in the order given: an ITO anode 5, a PEDOT hole-injection layer 10, an NPB hole-transport layer 15, a light-emitting layer 20, a TPBI electron-transport and hole-blocking layer 30, and a LiF/AI cathode 35.

For these particular examples, the ITO anode 5 was about 150 nm thick; the PEDOT hole injection layer 10 was about 30 nm thick; the NPB hole-transport layer 15 was about 40 nm thick; the light-emitting layer 20 was about 30 nm thick; the TPBI electron transport and hole blocking layer 30 was about 30 nm thick; the LiF sublayer (not shown) of the cathode 35 was about 0.5 nm thick; and the Al sublayer of the cathode (not shown) was about 120 nm thick. The device was then encapsulated with a getter attached glass cap to cover the emissive area of the OLED device in order to protect from moisture, oxidation or mechanical damage. Each individual device had an area of about 12 mm².

### Fabrication of Light-Emitting Devices:

### Device A

ITO substrates having sheet resistance of about 14 ohm/sq were cleaned ultrasonically and sequentially in detergent, water, acetone and then IPA; and then dried in an oven at 80° C. for about 30 min under ambient environment. Substrates were then baked at about 200° C. for about 1 hour in an ambient environment, then under UV-ozone treatment for about 30 minutes. PEDOT:PSS (hole-injection material) was then spin-coated on the annealed substrate at about 4000 rpm for about 30 sec. The coated layer was then baked at about 100° C. for 30 min in an ambient environment, followed by baking at 200° C. for 30 min inside a glove box (N₂ environment). The substrate was then transferred into a vacuum chamber, where 4,4′-bis[N-(naphthyl)-N-phenyl-amino]biphenyl (NPB) [hole transporting material]) was vacuum deposited at a rate of about 0.1 nm/s rate under a base pressure of about 2×10⁻⁷ torr. Bis(1-phenylisoquinoline)(acetylacetonate)iridium (III) (“Ir(piq)₂acac”) (10 wt %) was co-deposited as an emissive layer with Host-1 material at about 0.01 nm/s and about 0.10 nm/s, respectively, to make the appropriate thickness ratio. 1,3,5-Tris(1-phenyl-1H-benzimidazol-)2-yl)benzene (TPBI) was then deposited at about 0.1 nm/s rate on the emissive layer. A layer of lithium fluoride (LiF) (electron injection material) was deposited at about 0.005 nm/s rate followed by deposition of the cathode as Aluminum (Al) at about 0.3 nm/s rate.

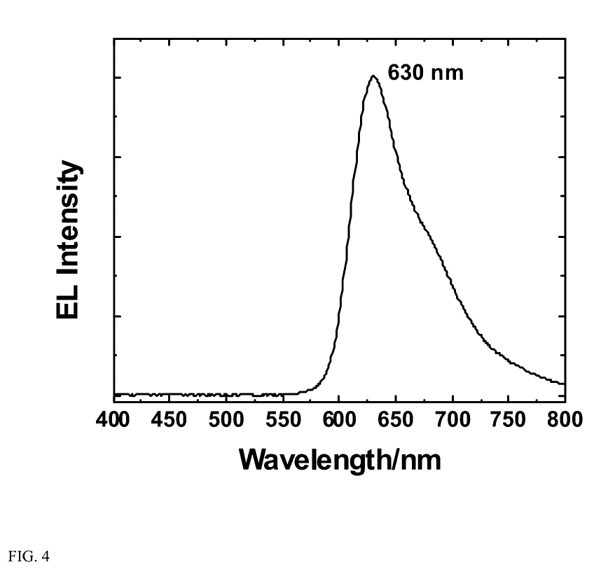

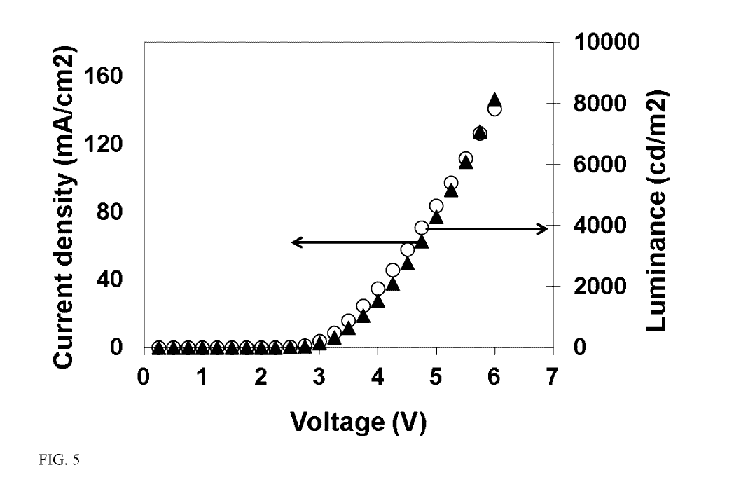

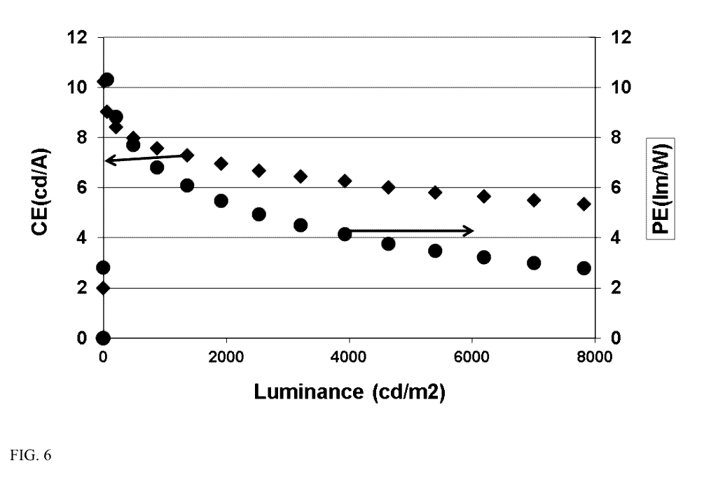

EL spectra, shown in FIG. 4, was measured with a Spectrascan spectroradiometer PR-670 (Photo Research, Inc., Chatsworth, Calif., USA); and I-V-L characteristics were taken with a Keithley 2612 SourceMeter (Keithley Instruments, Inc., Cleveland, Ohio, USA) and PR-670. In addition, device performance of the device was evaluated by measuring the current density and luminance as a function of the driving voltage, as shown in FIG. 5. FIG. 6 is a plot of current efficiency/power efficiency vs. luminance for the device. The turn-on voltage for the device was about 2.5 volts and the maximum luminance was about 39,700 cd/m² with 12 mm² area device at about 8V. The EQE (external quantum efficiency), luminous efficiency and power efficiency of the device at 1000 cd/m² were about 15.5%, 12.3 cd/A and 10.4 lm/w at 630 nm emission.

### Device B

Device B was fabricated in a manner similar to the following. The substrate (glass-SiON/Metal foil) was cleaned ultrasonically and sequentially in detergent, water, acetone and then IPA; and then dried in an oven at about 80° C. for about 30 min under ambient environment. Substrate was then baked at about 200° C. for about 1 hour under ambient environment, then under UV-ozone treatment for about 30 minutes. Soon after UV-ozone treatment, substrates were loaded into a deposition chamber. A bi-layer reflective type bottom anode, e.g., Al (about 50 nm) and Ag (about 40 nm) were deposited sequentially at a rate of about 0.1 nm/s. A hole injection layer as Dipyrazino[2,3-f:2′,3′-h]quinoxaline-2,3,6,7,10,11-hexacarbonitrile (HAT-CN, about 10 nm) was deposited as on reflective anode. NPB (about 40 nm) was then deposited as a hole-transport layer. Bis(1-phenylisoquinoline)(acetylacetonate)iridium (III) (“Ir(piq)₂acac”) (10 wt %) was co-deposited as an emissive layer with Host-1 material at about 0.01 nm/s and about 0.10 nm/s, respectively, to make the appropriate thickness ratio and a total thickness of about 20 nm. 1,3,5-Tris(1-phenyl-1H-benzimidazol-)2-yl)benzene (TPBI, about 50 nm) was then deposited at about 0.1 nm/s rate on the emissive layer. A thin layer of lithium fluoride (LiF, about 1 nm) (electron-injecting material) was deposited at about 0.005 nm/s rate, followed by deposition of the magnesium (Mg, about 1 nm) at about 0.005 nm/s rate. A semi-transparent cathode (about 16 nm) was deposited by co-deposition of magnesium (Mg) and silver (Ag) at a ratio of about 1:3 by weight. Finally a capping layer as NPB (about 60 nm) was deposited to enhance light output by micro cavity effect. All the deposition was done at a base pressure of about 2×10⁻⁷ torr.

Referring to FIG. 2, the first anode sublayer 7 was Al (about 50 nm thick), the second anode sublayer 9 was Ag (about 40 nm thick), the hole-injecting layer 10 was HAT-CN (about 10 nm thick), the hole-transport layer 15 was NPB (about 40 nm thick), the light-emitting layer 20 was Host-1: Ir(piq)₂acac (about 20 nm thick), the electron-transport layer 30 was TPBI (about 50 nm thick), the electron-injecting layer 25 was LiF (about 1 nm thick), the second cathode sublayer 38 was Mg (about 1 nm thick), the first cathode sublayer 37 was Mg:Ag (about 16 nm thick), and the capping layer 40 was NPB (about 60 nm thick). The device was then encapsulated with a getter attached clear glass cap to cover the emissive area of the OLED device in order to protect from moisture, oxidation or mechanical damage. In order to minimize heat effect for such large area device, a thermal compensating layer can be attached on the backside of the substrate with heat sink. This layer was a typical Al heat sink with fin structure. Other materials such as Cu-film and alloy films can also be used for similar purpose depending on the thermal conductivity of the materials. Each individual device has an area of about 1.8 cm².

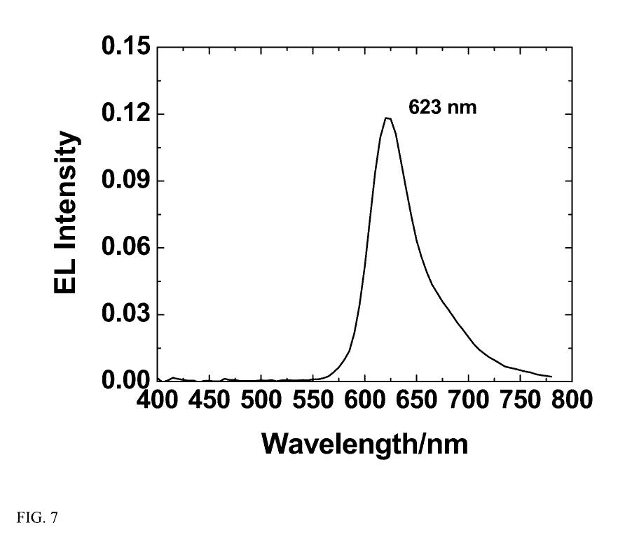

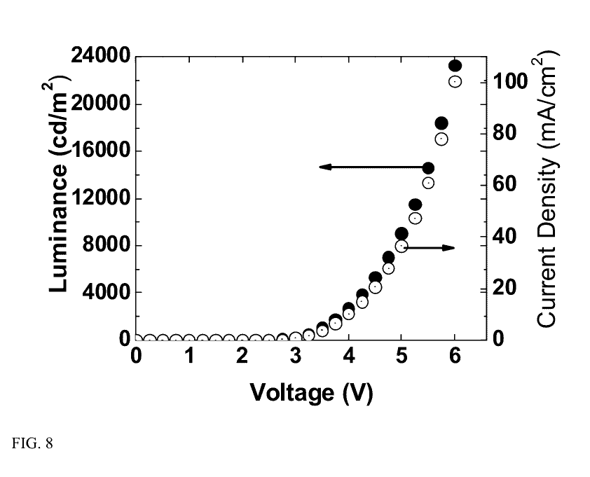

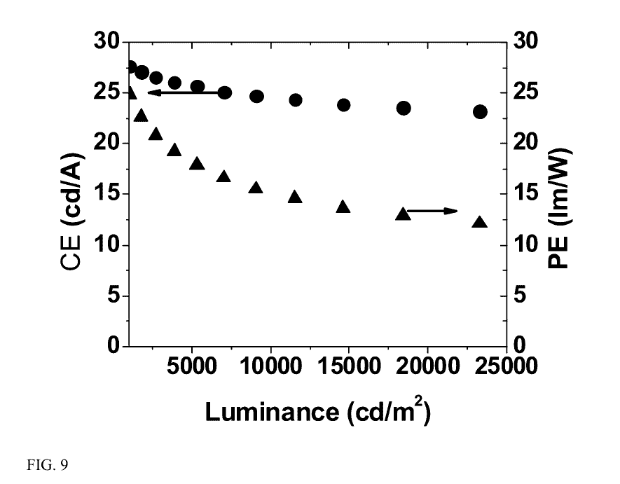

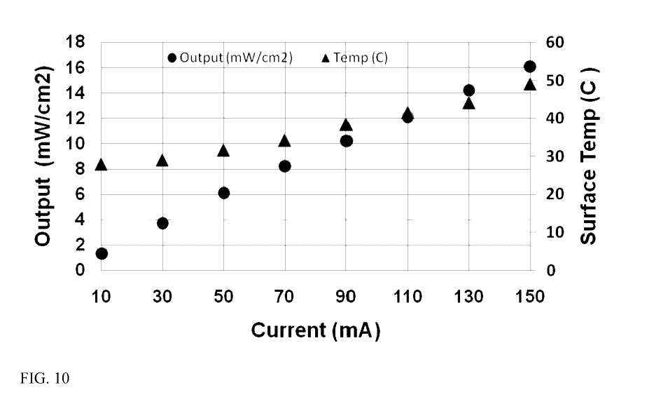

Performance of the Device B was evaluated. FIG. 7 is the electroluminescence spectrum of the device. FIG. 8 is a plot of luminance and current density as a function of applied voltage. The plot shows that the light power output of the device is sufficient for phototherapy at a voltage range that may be used for that application. FIG. 9 is a plot of current efficiency and power efficiency as a function of current density. FIG. 10 is a plot of light output as a function of input current. The plot shows that the light output can be 10 mW/cm² at 90 mA input current, which is sufficient for phototherapy at a luminance range that may be used for that application. The surface temperature is <40° C. and can be suitable to apply on the skin. The voltage required for 10 mW/cm² is about 5.5 V, which is suitable for operation with a portable and rechargeable battery. The turn-on voltage for the device was about 2.4 volts and the maximum luminance was about 24,500 cd/m² with 1.8 cm² area device at about 6V. At 1000 cd/m² intensity and 623 nm emission, the EQE (external quantum efficiency) was about 17.4%, luminous efficiency was about 27.6 cd/A, and the power efficiency of the device was about 24.8 lm/w.

### Example 3

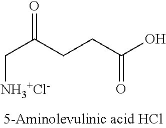

5-Aminolevulinic acid HCl (20% topical solution, available as LEVULAN® KERASTICK® from DUSA® Pharmaceuticals) is topically applied to individual lesions on a person suffering from actinic keratoses. About 14-18 hours after application, the treated lesions are illuminated with a red light-emitting OLED device constructed as set forth in Example 2.

After the treatment, the number or severity of the lesions is anticipated to be reduced. The treatment is repeated as needed.

### Example 4

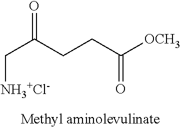

Methyl aminolevulinate (16.8% topical cream, available as METVIXIA® Cream from GALERMA LABORATORIES, Fort Worth, Tex., USA) is topically applied to individual lesions on a person suffering from actinic keratoses. The excess cream is removed with saline, and the lesions are illuminated with the red light-emitting OLED constructed as set forth in Example 2.

Nitrile gloves are worn at all times during the handling of methyl aminolevulinate. After the treatment, it is anticipated that the number or severity of the lesions is reduced. The treatment is repeated as needed.

### Example 5

Verteporphin is intravenously injected, over a period of about 10 minutes at a rate of about 3 mL/min, to a person suffering from age-related macular degeneration. The verteporphin (7.5 mL of 2 mg/mL reconstituted solution, available as Visudyne® from Novartis) is diluted with 5% dextrose to a volume of 30 mL using a sufficient quantity of the reconstituted verteporphin so that the total dose injected is about 6 mg/m² of body surface.

About 15 minutes after the start of the 10 minute infusion of verteporphin, the verteporphin is activated by illuminating the retina with a red light-emitting OLED device as set forth in Example 2.

After treatment, the patient's vision is anticipated to be stabilized. The treatment is repeated as needed.

### Example 6

Verteporphin is intravenously injected, over a period of about 10 minutes at a rate of about 3 mL/min, to a person suffering from pathological myopia. The verteporphin (7.5 mL of 2 mg/mL reconstituted solution, available as Visudyne® from Novartis) is diluted with 5% dextrose to a volume of 30 mL using a sufficient quantity of the reconstituted verteporphin so that the total dose injected is about 6 mg/m² of body surface.

About 15 minutes after the start of the 10 minute infusion of verteporphin, the verteporphin is activated by illuminating the retina with a red light-emitting OLED device as set forth in Example 2.

After treatment, the patient's vision is anticipated to be stabilized. The treatment is repeated as needed.

### Example 7

Verteporphin is intravenously injected, over a period of about 10 minutes at a rate of about 3 mL/min, to a person suffering from presumed ocular histoplasmosis. The verteporphin (7.5 mL of 2 mg/mL reconstituted solution, available as Visudyne® from Novartis) is diluted with 5% dextrose to a volume of 30 mL using a sufficient quantity of the reconstituted verteporphin so that the total dose injected is about 6 mg/m² of body surface.

About 15 minutes after the start of the 10 minute infusion of verteporphin, the verteporphin is activated by illuminating the retina with a red light-emitting OLED device (such as Device-A).

After treatment, the patient's vision is anticipated to be stabilized. The treatment is repeated as needed.

### Example 8

### Ex-Vivo Efficacy Study with Device A

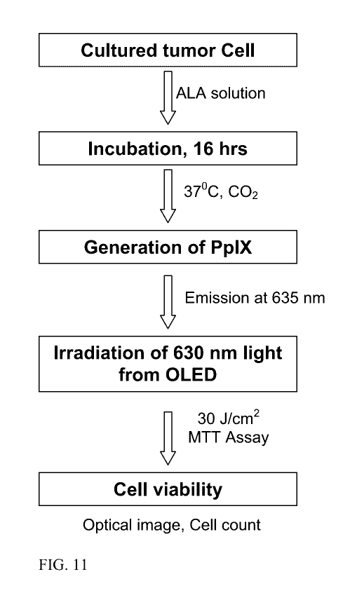

An efficacy study has been performed on CHO-K1 (Chinese Hamster Ovarian Cancer, ATCC, CRL-2243) cell line using the pro-drug 5-aminolevulinic acid (ALA). FIG. 11 exhibits the efficacy study scheme. Cells were cultured in a 96-well media (Hyclone F-12K medium and dulbeccdo phosphate buffer saline, DPBS) and incubated at 37° C. under a CO₂ atmosphere for about 24 hrs. The cells were calibrated by cell counting with a standard cross area under optical microscope (Olympus IX-70) to establish a base reference number of cells of about 10,000 counts in 100 uL medium per well plate. ALA solutions (0.84 mg/mL˜3.3 mg/mL in F-12K medium) with three different concentrations: 0.5 mM, 1 mM, and 2 mM, were introduced into same media as mentioned above and incubated for about 16 hrs at 37° C. under a CO₂ atmosphere. While not being limited by theory, it is believed that in this process, ALA undergoes a biological transformation and is converted to protoporphyrin IX (PpIX). The generation of PpIX was confirmed by fluorescence emission at 635 nm.

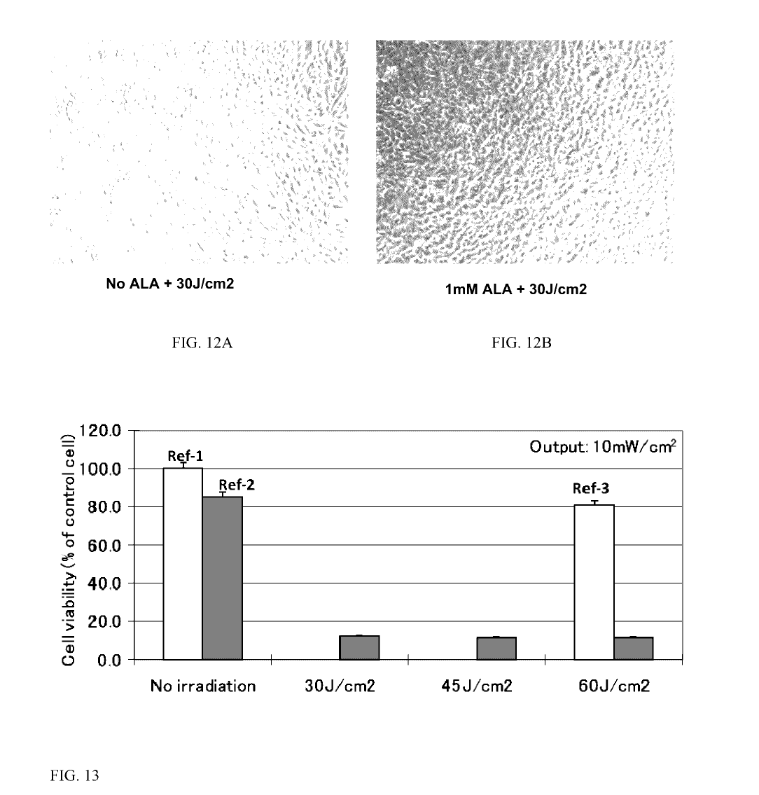

An OLED was constructed similar to those of Example 2 (emissive layer comprising Compound X:Ir(piq)₂acac) (Device B). Red light (623 nm) was then generated by the OLED to provide a total dose of about 30 to 60 J/cm² with output power of 10 mW/cm². While not being limited by theory, it is believed PpIX absorbs 630 nm light and is excited to its singlet state followed by intersystem crossing to triplet state. While not being limited by theory, it is believed that since the triplet state may have a longer lifetime, the triplet PpIX may interact with molecular oxygen and may generate singlet oxygen and other reactive oxygen species (ROS). These ROS may have a shorter lifetime and may have a diffusion length of only about several tens of nm. The ROS within their area may then undergo cytotoxic reaction with different cell components such as cell membrane, mitochondria, lissome, golgy bodies, nucleus etc and may destroy them and ultimately tumor cell dies. Optical microscope (Olympus IX-70) images of the cells after about 30 J/cm² red light irradiation shows (FIG. 12) that the healthy leafy type cells (FIG. 12A) transforms to droplet type upon light irradiation (FIG. 12B) indicating a significant and irreversible cell death.

Following light irradiation, 10 uL of MTT solution (Invitrogen, 3, (4,5-dimethylthiazol-2-yl)-2,5-diphenyl tetrazolium bromide, 5 mg/mL in DPBS) was added to each well, including the control well, and shaken well to mix thoroughly. The wells were incubated (37° C., 5% CO₂) for 1.5 hrs to generate purple crystals. Then 100 uL MTT solubilization solutions were added to each well and incubated (37° C., 5% CO₂) for 16 hrs to dissolve the purple crystals. Finally the absorbance of the cells at 570 nm with a reference wavelength at 690 nm were recorded by a microplate reader (BioTeK MQX-200) in order to estimate cell viability (%). Cell viability results are shown in FIG. 13. At ALA concentrations of about 1 mM or higher, almost 90% of cells were destroyed as compared to the reference cells. Reference cells were irradiated with same dose of light but without ALA, or kept in a normal environment without light irradiation.

Light Dosimetry was used to optimize the irradiation dose. FIG. 13 shows the cell viability results compared with the references. Reference 1 corresponds to the cells in a solution without ALA and exposed to outdoor light for an identical period of time to that of OLED irradiation for the test samples. Reference-2 corresponds to the cells in a solution having a 1 mM ALA concentration and exposed to outdoor light in the same way as ref-1. However, reference-3 corresponds to the cells in a solution without ALA that was irradiated with a 60 J/cm² dose from an OLED. Reference-3 was used to as comparison in order to determine whether there was any cell damage by heat generated from the OLED. Cells remained alive in all reference samples.

These references can be compared with the experimental cells where the concentration of ALA was fixed at 1 mM and light output was fixed at 10 mW/cm². The light doses were varied from 30 J/cm² to 60 J/cm² by varying the time of exposure to the light. As shown, almost 90% cells were destroyed with a light dose above about 30 J/cm², indicating that the OLED has potential for use as a light source for PDT treatment. A light dose of about 30 J/cm² takes about 50 minutes to administer at a power output of 10 mW/cm². However, higher output power may allow the same dose, e.g. 30 J/cm², to be administered in less irradiation time.

### Example 9

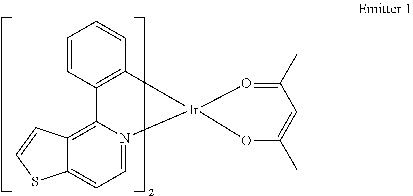

Four devices were prepared with the following structure: glass substrate (700 μm)/Si₃N₄ (40 nm)/HAT-CN (10 nm)/NPB (50 nm)/compound X: Emitter 1 (20 nm) (10 wt %)/TPBI (50 nm)/LiF (1 nm)/Mg: Ag (20 nm) (1:3)/MoO₃ (60 nm).

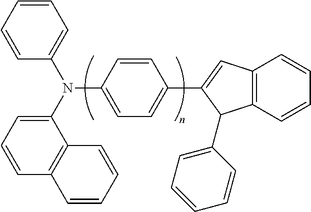

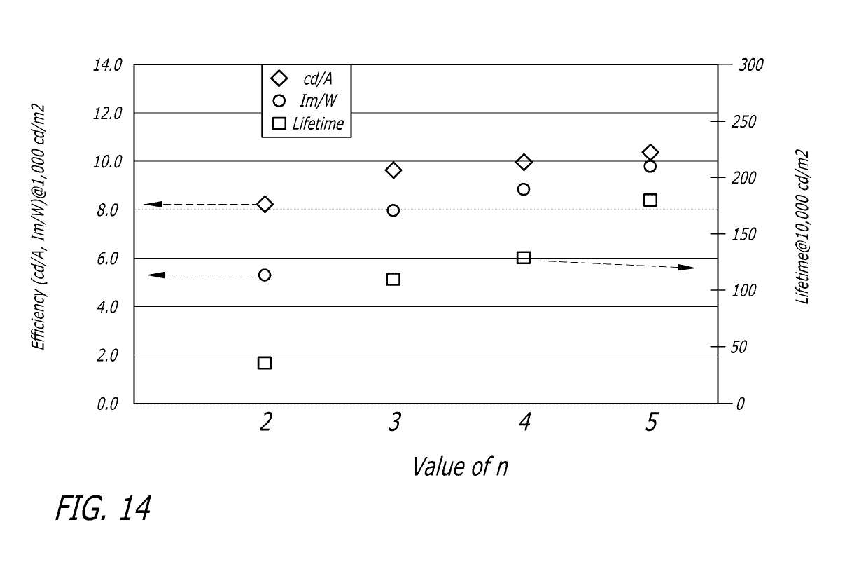

For each device, n of compound X was 2, 3, 4, or 5. The efficiency and lifetime of these devices is plotted as a function of the value n in FIG. 14.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

What is claimed is:

1. A light-emitting device for use in phototherapy comprising:
a light-emitting layer comprising a compound selected from the group consisting of: 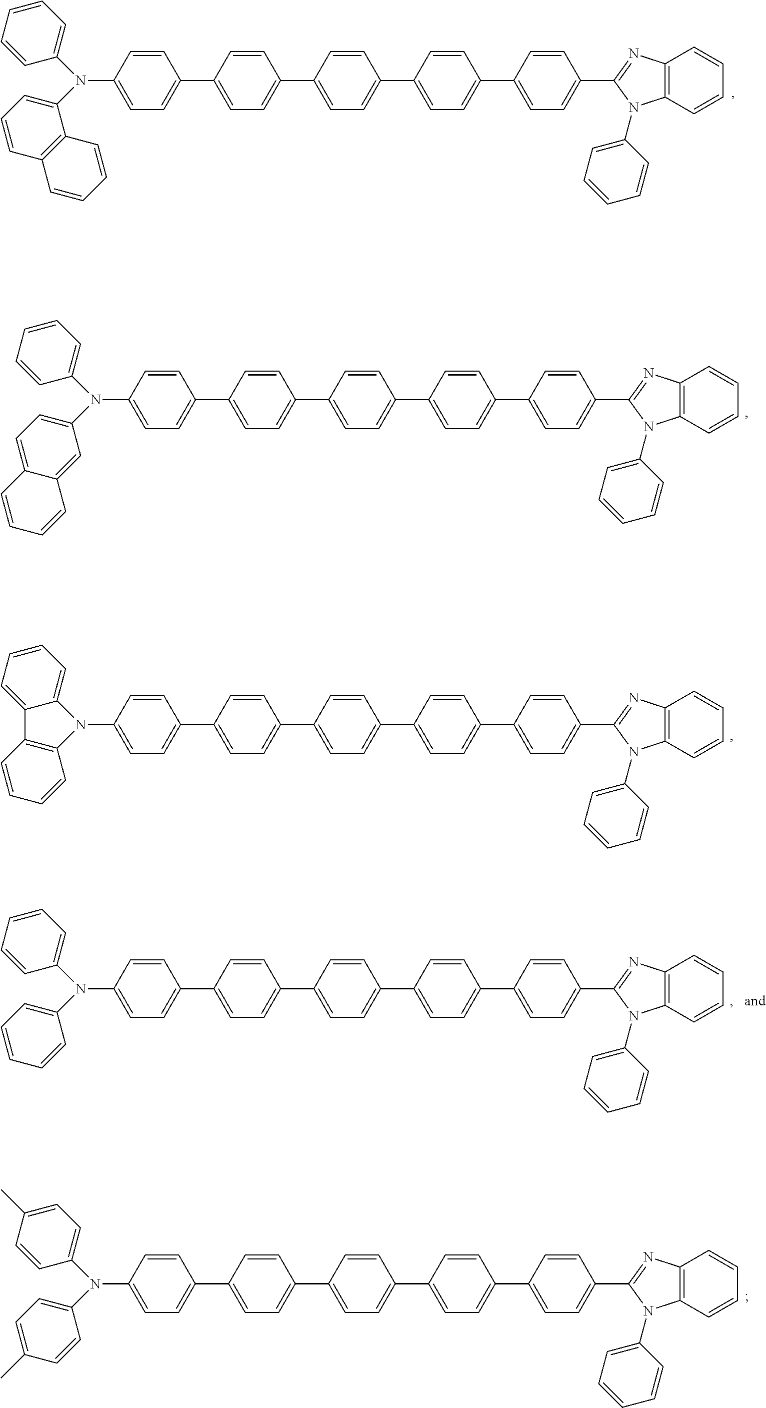
wherein the device is configured to emit an effective amount of light to provide a therapeutic effect to a mammal, wherein the therapeutic effect is provided by contact between the emitted light and the mammal;
wherein the light has a wavelength that can activate at least a portion of a photosensitive compound which has been administered to a tissue of the mammal;
wherein the device further comprises a dosage component configured to control the amount of light emitted so that the tissue receives a light dose that is in the range of about 10 Joules/cm² to about 200 Joules/cm².

2. The device of claim 1, wherein the light has a wavelength that can activate at least a portion of a photosensitive compound which has been administered to a tissue of the mammal.

3. The device of claim 1, wherein the dosage component comprises a timer.

4. A phototherapy system comprising:
a device according to claim 1; and
a wound dressing.

5. The phototherapy system of claim 4, further comprising a dosage component configured to control the amount of light emitted, wherein the amount of light emitted is effective to provide a therapeutic effect for treating a disease.

6. The phototherapy system of claim 4, wherein the device comprises:

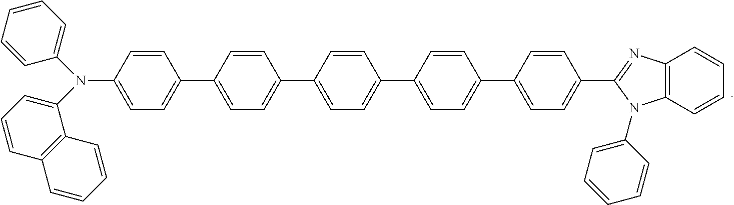 [SMILES: c1ccc(N(c2ccc(-c3ccc(-c4ccc(-c5ccc(-c6ccc(-c7nc8ccccc8n7-c7ccccc7)cc6)cc5)cc4)cc3)cc2)c2cccc3ccccc23)cc1]

7. The phototherapy system of claim 4, wherein the device comprises:

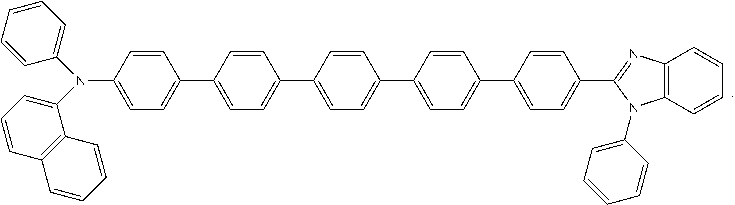 [SMILES: c1ccc(N(c2ccc(-c3ccc(-c4ccc(-c5ccc(-c6ccc(-c7nc8ccccc8n7-c7ccccc7)cc6)cc5)cc4)cc3)cc2)c2cccc3ccccc23)cc1]

8. The phototherapy system of claim 4, wherein the device comprises:

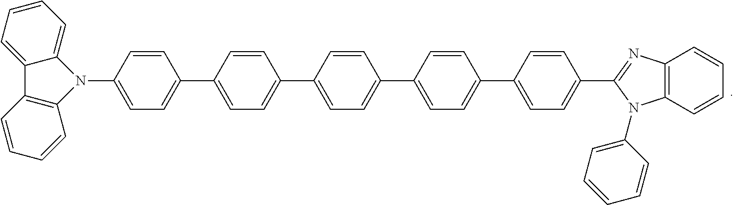 [SMILES: c1ccc(-n2c(-c3ccc(-c4ccc(-c5ccc(-c6ccc(-c7ccc(-n8c9ccccc9c9ccccc98)cc7)cc6)cc5)cc4)cc3)nc3ccccc32)cc1]

9. The phototherapy system of claim 4, wherein the device comprises:

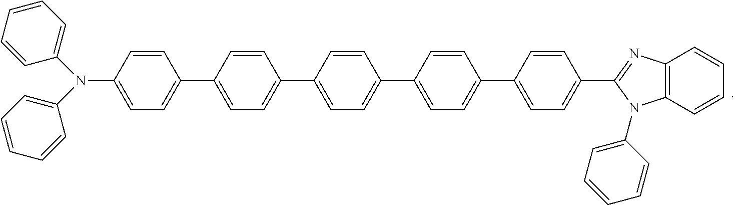 [SMILES: c1ccc(N(c2ccccc2)c2ccc(-c3ccc(-c4ccc(-c5ccc(-c6ccc(-c7nc8ccccc8n7-c7ccccc7)cc6)cc5)cc4)cc3)cc2)cc1]

10. The phototherapy system of claim 4, wherein the device comprises:

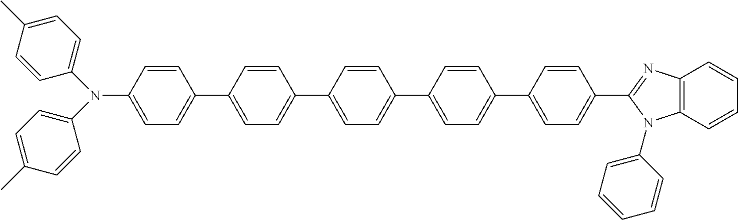 [SMILES: Cc1ccc(N(c2ccc(C)cc2)c2ccc(-c3ccc(-c4ccc(-c5ccc(-c6ccc(-c7nc8ccccc8n7-c7ccccc7)cc6)cc5)cc4)cc3)cc2)cc1]

11. A method of carrying out phototherapy comprising:
exposing at least a portion of a tissue of a mammal to light from the device of claim 1.

12. The method of claim 11, further comprising administering a photosensitive compound to the tissue, wherein at least a portion of the photosensitive compound is activated by light from the device.

13. A method of treating a disease, comprising:
exposing at least a portion of a tissue of a mammal in need thereof to light from the device of claim;
wherein the tissue contains a photosensitive compound; and
wherein at least a portion of the photosensitive compound is activated by at least a portion of the light from the device to which the tissue is exposed, to thereby treat the disease.

14. The method of claim 12, wherein activating the photosensitive compound produces singlet oxygen.

15. The method of claim 12, wherein the photosensitive compound is 5-aminolevulinic acid, methyl aminolevulinate, verteporfin, zinc phthalocyanine, or pharmaceutically acceptable salts thereof.

16. The method of claim 15, wherein the photosensitive compound is 5-aminolevulinic acid.

17. The method of claim 16, wherein the 5-aminolevulinic acid is present in the tissue at a concentration of about 0.5 to about 2 mM.

18. The method of claim 17, wherein the device has a peak emission of about 630 nm.

19. The method of claim 18, wherein the tissue receives a light dose that is at least about 30 Joules/cm².

20. The method of claim 19, wherein the tissue receives a light dose that is in the range of about 30 Joules/cm² to about 60 Joules/cm².

21. The method of claim 13, wherein the disease is a cancer.

22. The method of claim 13, wherein the disease is a microbial infection.

23. The method of claim 13, wherein the disease is a dermatological condition.

24. The method of claim 13, wherein the disease is an eye condition.

25. A phototherapy system comprising:
a device according to claim 1; and
a photosensitive compound;
wherein the photosensitive compound is suitable for administration to a tissue of a mammal in need of phototherapy; and
wherein the device is configured to emit light of a wavelength which can activate the photosensitive compound when the photosensitive compound is in the tissue.

26. The phototherapy system of claim 25, wherein the photosensitive compound is 5-aminolevulinic acid, verteporfin, zinc phthalocyanine, or pharmaceutically acceptable salts thereof.

27. The method of claim 11, wherein the light is delivered in a pulsed manner.

28. The method of claim 11, wherein the light is delivered in a non-pulsed manner.
